SEQUENCE LISTING (43 sequences)

SEQ ID NO: 1 - DNA, 23 bp, synthetic construct
  source 1..23: mol_type = other DNA
taatacgactcactataggggaa

SEQ ID NO: 2 - DNA, 23 bp, synthetic construct
  source 1..23: mol_type = other DNA
taatacgactcactatcaaggaa

SEQ ID NO: 3 - DNA, 23 bp, synthetic construct
  source 1..23: mol_type = other DNA
taatacgactcactatttcggaa

SEQ ID NO: 4 - DNA, 23 bp, synthetic construct
  source 1..23: mol_type = other DNA
taatacgactcactaatactgaa

SEQ ID NO: 5 - DNA, 23 bp, synthetic construct
  source 1..23: mol_type = other DNA
taatacgactcactacggaagaa

SEQ ID NO: 6 - DNA, 101 bp, synthetic construct
  source 1..101: mol_type = other DNA
gcgtaatgcttaggcacaggattgatttgtcgcaatgattgacacgattccgcttgacgctgcgtaaggtttttgtaattttacaggcaaccttttattca

SEQ ID NO: 7 - DNA, 90 bp, synthetic construct
  source 1..90: mol_type = other DNA
ttgaaataaacatttattgtatatgatgagataaagttagtttattggataaacaaactaactcaattaagatagttgatggataaactt

SEQ ID NO: 8 - DNA, 20 bp, synthetic construct
  source 1..20: mol_type = other DNA
cggctccatatgcactctcg

SEQ ID NO: 9 - DNA, 20 bp, synthetic construct
  source 1..20: mol_type = other DNA
gagccgctcgagttaactgg

SEQ ID NO: 10 - DNA, 21 bp, synthetic construct
  source 1..21: mol_type = other DNA
cggctccatatgcacatcagg

SEQ ID NO: 11 - DNA, 23 bp, synthetic construct
  source 1..23: mol_type = other DNA
gagccgctcgagctagaacaaag

SEQ ID NO: 12 - DNA, 25 bp, synthetic construct
  source 1..25: mol_type = other DNA
cggctccatatgcacaacagaaacc

SEQ ID NO: 13 - DNA, 22 bp, synthetic construct
  source 1..22: mol_type = other DNA
gagccgctcgagtcagacaaag

SEQ ID NO: 14 - DNA, 25 bp, synthetic construct
  source 1..25: mol_type = other DNA
cggctccatatgcatatcaggaacc

SEQ ID NO: 15 - DNA, 23 bp, synthetic construct
  source 1..23: mol_type = other DNA
gagccgctcgagctagaaaagag

SEQ ID NO: 16 - DNA, 37 bp, synthetic construct
  source 1..37: mol_type = other DNA
gccgcccatatgcatatcagaaatccttaccgtacac

SEQ ID NO: 17 - DNA, 36 bp, synthetic construct
  source 1..36: mol_type = other DNA
ggcgcgactagtctagaaaagagagctgagctcggg

SEQ ID NO: 18 - protein, 430 aa, synthetic construct
MAKTLRPTAQAFRELGWLPASDGVYNKFMKDLTNRASNENHLCHVALLQPIQDFKTFIENDPVVYQEFVCMFEGIEESPRNYHELCNMFNEIFRRAPYYGDLGPPVYMAMAKIMNTRAGFSAFTRESLNFHFKRLFDTWGLFLSSPASRDVLVADKFDSKHYGWFSEPAKAAMMAQYDGRTFEQVFICDETAPYHGFKSYDDFFNRKFRAMDIDRPVVGGIANTTLIGSPCEALSYNVSDDVHSLETLYFKGEGYSLRHLLHDDPSTEQFEHGSIIQGFLNITGYHRWHAPVSGTIMKIVDVPGTYFAQAPSTIGDPFPVNDYDPQAPYLRSLAYFSNIAARQIIFIQADNEDIGLIYLILIGMTEVSTCEALVCPGQHVERGDDLGMFHFGGSSFALGLRKNSKAAILEELKTQGTVIKVNDVIAAVQA

SEQ ID NO: 19 - DNA, 1293 bp, synthetic construct
  source 1..1293: mol_type = other DNA
atggccaaaacgctacgacccactgcccaggcctttcgagaactcggttggctgcctgccagcgacggagtttacaacaagttcatgaaggacttgacgaatcgggccagcaacgaaaatcacttatgccatgttgcccttctgcagcccatccaagatttcaaaacattcattgagaacgatcctgttgtgtaccaggaatttgtttgcatgtttgagggaatcgaggagtctcctagaaattatcatgagctatgtaacatgttcaacgaaatcttccgaagggccccatattacggggatctagggcctccagtgtacatggccatggctaaaattatgaatacgcgagctggcttctccgcattcacaagagagagcttgaacttccacttcaaaagactcttcgatacttggggtttattcctttcctcgccagcctcacgcgacgtgcttgttgcagacaagttcgacagcaagcattatggctggtttagcgaacctgccaaggcggctatgatggctcaatacgacggacgtacatttgaacaagtctttatctgcgacgagaccgctccttaccacggcttcaaatcttacgacgactttttcaaccggaaattcagagccatggacatcgatcgcccagtcgtcggtgggatcgccaacactaccctcattgggtctccttgcgaagcgttgtcgtacaacgtctcggatgacgtccattctttggaaactctgtacttcaaaggcgagggttattctctcagacacctgctccacgacgatccttctacggaacagttcgagcatggaagtattattcaaggattcctcaacatcactggctatcaccgatggcacgcacccgtgagtggaacaatcatgaagatcgtcgacgtcccgggcacctacttcgctcaggcgcccagcacaattggagatccattcccagtcaatgactacgacccgcaggctccttacctcaggtctctcgcatacttctccaacattgccgccaggcagattatcttcatccaagccgacaacgaggacatcggcttgatatatctaattctaatcggtatgacggaggtctcgacttgcgaggcccttgtgtgccctggtcagcatgtcgaacggggcgacgatctgggaatgttccatttcggtggttcatccttcgctcttggccttcgcaagaactcaaaagccgcgattctcgaagaactcaagacgcagggaactgtcatcaaagtcaacgacgtgatagcggctgttcaagcgtaa

SEQ ID NO: 20 - protein, 360 aa, synthetic construct
MTFDLKTEEGLLVYLTQHLSLDVDLDGLKRLSGGFVNITWRIRLNAPFKGYTNIILKHAQPHLSSDENFKIGVERSAYEYRALKIVSESPILSGDDNLVFVPQSLHYDVVHNALIVQDVGSLKTLMDYVTARPSLSSEMAKLVGGQIGAFIARLHNIGRENKDHPEFNFFSGNIVGRTTAVQLYETIVPNATKYDIDDPIIPVVVQELIEEVKGSDETLIMADLWGGNILLEFGKDSSDLGKIWVVDWELCKYGPPSLDMGYFLGDCFLLAQFQDEKVATAMRRAYLENYAKIAKVPMDYDRSTTGIGAHLVMWTDFMNWGSDEERKTSVEKGVRAFHDAKRDNKEGEIPSILLRESSRT

SEQ ID NO: 21 - DNA, 1083 bp, synthetic construct
  source 1..1083: mol_type = other DNA
atgactttcgatctcaagactgaagaaggcctcttagtctatcttactcagcacctatcgttggacgtcgacctcgatgggctgaagcgtctcagcggcggcttcgtcaacatcacctggcggattagactcaacgctcctttcaaaggttacacgaacatcatcttgaagcacgctcagccgcacttatcgtcagacgagaattttaagattggggtagagcggtcggcatacgaatatcgagcactgaaaatcgtgtctgagagtcctatacttagcggcgatgataatcttgtcttcgtacctcaaagtcttcattacgacgtcgttcataatgccttgatcgtgcaagacgtggggtcgctgaagaccctcatggattatgtcacggccagaccgtcactttcatcggagatggccaagcttgtcggcggtcagattggtgccttcatcgctcgactgcataatatcggacgcgagaataaggaccatccggaattcaattttttctcgggaaacatcgtcggaagaacaacggctgttcagctatatgaaaccatcgttcccaacgccaccaagtacgatatcgacgacccgattattcctgtagtggttcaggagttgatcgaggaagtcaaaggcagcgacgagacgcttataatggcggatctgtggggtggcaatatccttctcgagtttgggaaggactcctcggatttgggaaagatatgggtcgtagactgggagttatgcaaatacggacccccttctttggacatgggttacttcttaggcgattgtttccttctcgctcagtttcaagacgaaaaggtcgcgacggccatgagaagggcctacttggagaattacgcgaagattgccaaggtcccaatggactatgataggagcacgacaggcattggggcgcatctcgtcatgtggactgacttcatgaattgggggagcgatgaggaaaggaagacgtctgtggagaagggtgtcagggctttccatgatgcaaagagggacaacaaggaaggggaaattccatctatacttttgcgagaatcgtcaagaacgtag

SEQ ID NO: 22 - protein, 309 aa, synthetic construct
MHIRNPYLTPPDYEALAEAFPALKPYVTVNPDKTTTIDFAIPEAQRLYTAALLYRDFGLTITLPPDRLCPTVPNRLNYVLWIQDILQITSAALGLPEARQVKGVDIGTGAAAIYPILGCSLAKNWSMVGTEVEQKCIDIARQNVISNGLQDRITITANTIDAPILLPLFEGDSNFEWEFTMCNPPFYDGAADMETSQDAKGFGFGVNAPHTGTVVEMATDGGEAAFVSQMVRESLHLKTRCRWFTSNLGKLKSLHEIVGLLREHQITNYAINEYVQGTTRRYAIAWSFTDLRLSDHLPRPPNPDLSALF

SEQ ID NO: 23 - DNA, 930 bp, synthetic construct
  source 1..930: mol_type = other DNA
atgcacatcaggaatccgtacctcactcccccagactacgaagccttggctgaagcatttccagccctcaagccatacgtgacggtcaatcctgacaagacgaccactatcgatttcgcaataccagaagctcaaagactgtatacagcagccctcctctaccgcgatttcggtttgacaatcacactacccccagatcgtttgtgtccaacggtgcccaatcggctcaactacgtcctctggatccaggatatccttcaaatcacttctgctgctctaggtctccccgaggcacgtcaggtcaaaggggttgatatcgggaccggtgcagcagcaatttatcccatcctcggttgctctctggccaagaactggtccatggttggaacagaagtagaacagaagtgcattgacatagcgcgccagaacgtcatatctaacgggctccaagatcgtatcacgataacggccaacaccatcgatgcgcctatccttctcccactttttgagggcgactctaactttgagtgggagttcaccatgtgtaatccgcctttttacgacggagccgccgacatggagacgtcgcaggatgcgaaaggctttgggttcggagtgaatgctccacatacagggacagttgtcgagatggccactgatggaggcgaagccgctttcgtgagccagatggttcgcgaaagtctccatcttaaaacacgctgcaggtggttcacgagtaacttgggaaagctgaagtctcttcatgaaattgtggggctcttacgcgaacatcagataaccaactacgcaatcaatgaatatgtccaaggaactacacgccgttacgcaattgcgtggtcgttcaccgaccttcgccttagcgatcatttgcctcgcccccctaatcccgatttgagtgctttgttctag

SEQ ID NO: 24 - protein, 297 aa, synthetic construct
MHSRNPYRSPPDFAALSAAYPPLSPYITTDLSSGRKTIDFRNEEAQRRLTEAIMLRDFGVVLNIPSNRLCPPVPNRMNYVLWIQDIVYAHQTILGVSSRRIRGLDIGTGATAIYPILACKKEQSWEMVATELDDYSYECACDNVSSNNMQTSIKVKKASVDGPILFPVENQNFDFSMCNPPFYGSKEEVAQSAESKELPPNAVCTGAEIEMIFSQGGEEGFVGRMVEESERLQTRCKWYTSMLGKMSSVSTIVQALRARSIMNYALTEFVQGQTRRWAIAWSFSDTHLPDAVSRISS

SEQ ID NO: 25 - DNA, 894 bp, synthetic construct
  source 1..894: mol_type = other DNA
atgcactctcgtaacccttatagatcccctcctgatttcgcggcattaagtgcggcttatcctccgctgtcaccatacataactaccgatctaagcagcggtcgtaaaacaattgactttagaaatgaggaagcgcaacgtcgtctaactgaggctatcatgttgcgtgacttcggcgttgtgttaaacataccatctaacaggctgtgcccgcctgtgccgaatcgtatgaactatgtactttggatacaagatatagtttacgcgcaccagacaatactgggagtgagttctcgtcgtatcagaggtcttgatattggtactggtgctaccgctatatatcctatactggcatgcaagaaagagcagagctgggagatggttgcaactgaattggacgactactcctatgagtgtgcatgtgataacgtgtcatccaacaatatgcagacttccattaaagtaaagaaggcttcggtagatgggccgatcctgttcccagtggaaaaccaaaatttcgactttagcatgtgcaacccgcctttctacggctctaaggaggaggtggcgcaatccgcagagtcaaaagaactgccgcccaatgctgtttgcacgggtgcagagatcgagatgatatttagtcaaggaggagaagagggtttcgtaggtagaatggtagaggaatcagagaggttgcaaacgagatgcaaatggtacacttcaatgcttggtaagatgtctagtgtaagcactatagttcaggctctgcgtgcgagatcaattatgaattatgctttgacagaatttgtacaaggacaaacccgtaggtgggcgatagcttggtctttctccgacactcacttaccggatgccgtcagtagaatctccagttaa

SEQ ID NO: 26 - protein, 439 aa, synthetic construct
MQVLPACQSSALKTLCPSPEAFRKLGWLPTSDEVYNEFIDDLTGRTCNEKYSSQVTLLKPIQDFKTFIENDPIVYQEFISMFEGIEQSPTNYHELCNMFNDIFRKAPLYGDLGPPVYMIMARIMNTQAGFSAFTKESLNFHFKKLFDTWGLFLSSKNSRNVLVADQFDDKHYGWFSERAKTAMMINYPGRTFEKVFICDEHVPYHGFTSYDDFFNRRFRDKDTDRPVVGGVTDTTLIGAACESLSYNVSHNVQSLDTLVIKGEAYSLKHLLHNDPFTPQFEHGSIIQGFLNVTAYHRWHSPVNGTIVKIVNVPGTYFAQAPYTIGSPIPDNDRDPPPYLKSLVYFSNIAARQIMFIEADNKDIGLIFLVFIGMTEISTCEATVCEGQHVNRGDDLGMFHFGGSSFALGLRKDSKAKILEKFAKPGTVIRINELVASVRK

SEQ ID NO: 27 - DNA, 1320 bp, synthetic construct
  source 1..1320: mol_type = other DNA
atgcaggtactgcccgcgtgccaatcttccgcgcttaaaacattgtgcccatcccccgaggcctttcgaaagctcggttggctccctactagcgacgaggtttacaacgaattcatcgatgacttgaccggtcgcacgtgcaatgaaaagtactccagccaggttacacttttgaagcctatccaagatttcaagacattcatcgagaatgatcccatagtgtatcaagaatttatctctatgtttgaaggaatcgagcagtctcccaccaactaccacgagctatgtaacatgttcaacgacatctttcgcaaagccccactctacggcgatcttggtcctccggtttacatgatcatggccagaataatgaatacgcaggcgggtttctctgcgttcacaaaagagagcttgaacttccatttcaaaaagctcttcgacacctgggggctattcctttcctcgaaaaactctcgaaacgtgcttgttgcagaccagtttgacgataagcattacgggtggttcagcgagcgagccaagactgccatgatgattaattatccagggcgtacattcgagaaagtcttcatctgcgacgagcacgttccataccatggcttcacttcctatgacgatttcttcaatcgcaggttcagggacaaggatacagatcggcccgtagtcggtggggttactgacaccactttaatcggggctgcctgtgaatcgttgtcatataacgtctctcacaacgtccagtctcttgacacgctagtcatcaagggagaggcctattcacttaaacatctacttcataacgaccccttcacaccgcaattcgaacatgggagcatcattcaaggattcctaaatgtcaccgcttaccaccgctggcactcccccgtcaatggcacgattgtgaagatcgtcaacgttccaggtacctacttcgctcaagctccatatacaattggatctcctatccccgataacgaccgcgacccgcctccttacctcaagtcactcgtatacttctccaacatcgctgcacggcaaattatgttcatcgaggccgacaacaaagacatcggcctcattttcttggtcttcattggaatgactgagatctcgacttgcgaggcgacggtgtgcgaaggtcagcatgtcaaccgcggtgacgatttgggcatgttccatttcggtggttcatcttttgcccttggcttgcggaaggactcgaaggcgaagattttggaaaagttcgcgaaaccggggaccgttattaggatcaacgagctagttgcatctgtaaggaagtag

SEQ ID NO: 28 - protein, 361 aa, synthetic construct
MTFDLKTEEGLLSYLTKHLSLDVAPNGVKRLSGGFVNVTWRVGLNAPYHGHTSIILKHAQPHLSSDIDFKIGVERSAYEYQALKIVSANSSLLGSSDIRVSVPEGLHYDVVNNALIMQDVGTMKTLLDYVTAKPPISAEIASLVGSQIGAFIARLHNLGRENKDKDDFKFFSGNIVGRTTADQLYQTIIPNAAKYGIDDPILPIVVKELVEEVMNSEETLIMADLWSGNILLQFDENSTELTRIWLVDWELCKYGPPSLDMGYFLGDCFLVARFQDQLVGTSMRQAYLKSYARNVKEPINYAKATAGIGAHLVMWTDFMKWGNDEEREEFVKKGVEAFHEANEDNRNGEITSILVKEASRT

SEQ ID NO: 29 - DNA, 1086 bp, synthetic construct
  source 1..1086: mol_type = other DNA
atgactttcgatctcaagactgaagaaggcctgctctcatacctcacaaagcacctatcgctggacgttgctcccaacggggtgaaacgtcttagtggaggcttcgtcaacgttacctggcgggtcgggctcaatgccccttatcatggtcacacgagcattattctgaagcatgctcaaccgcacctgtcttcagacatagatttcaagataggtgttgaacgatcggcgtacgagtatcaagcgctcaaaatcgtgtcagccaatagctcccttctaggcagcagcgatattcgggtctctgtaccagaaggtcttcactacgacgtcgttaataacgcattgatcatgcaagatgtcgggacaatgaagaccctgttggactatgtcactgccaaaccaccaatttctgcagagatcgccagtctcgtaggcagtcaaattggtgcatttatcgctaggctgcacaacctcggccgcgagaataaagacaaggacgacttcaagttcttctctggaaacatcgtcgggagaacaaccgcagaccagttgtatcaaaccatcatacctaatgccgctaaatacggtatcgacgatccaattctcccaattgtggtaaaggagttggtggaggaggtcatgaatagtgaagaaacgcttatcatggcggatttatggagtggcaatattcttctccagtttgatgaaaactcgacggaattgacgaggatatggctggtagactgggagttgtgcaaatatggtccaccgtctttggacatggggtacttcttaggcgactgtttcctggtcgctcgatttcaagatcagctcgtagggacatcaatgcgacaggcctacttgaagagctacgcaaggaatgtcaaggagccaatcaattatgcaaaagccaccgcaggcatcggcgcgcatctcgtcatgtggactgatttcatgaagtgggggaacgatgaagagagggaagagtttgttaagaaaggcgtggaagccttccatgaagcaaatgaggacaatagaaacggggagattacgtctatacttgtgaaggaagcatcgcgcacttag

SEQ ID NO: 30 - protein, 309 aa, synthetic construct
MHIRNPYRDGVDYQALAEAFPALKPHVTVNSDNTTSIDFAVPEAQRLYTAALLHRDFGLTITLPEDRLCPTVPNRLNYVLWVEDILKVTSDALGLPDNRQVKGIDIGTGASAIYPMLACSRFKTWSMVATEVDQKCIDTARLNVIANNLQERLAIIATSVDGPILVPLLQANSDFEYDFTMCNPPFYDGASDMQTSDAAKGFGFGVNAPHTGTVLEMATEGGESAFVAQMVRESLNLQTRCRWFTSNLGKLKSLYEIVGLLREHQISNYAINEYVQGATRRYAIAWSFIDVRLPDHLSRPSNPDLSSLF

SEQ ID NO: 31 - DNA, 930 bp, synthetic construct
  source 1..930: mol_type = other DNA
atgcatatcaggaacccataccgcgatggtgttgactaccaagcactcgctgaagcatttccggctctcaaaccacatgtcacagtaaattcagacaatacgacctccatcgactttgctgtgccagaagcccaaagactgtatacagctgcccttctacaccgggatttcggtcttacgatcacactcccggaagaccgtctttgtccgacagtgcctaatcggctcaactatgtcctttgggttgaagatatccttaaagtcacttctgatgctctcggtcttccggataatcgtcaagttaaggggatcgatatcggaactggcgcatcagcgatatatcccatgctcgcatgctctcgttttaagacatggtccatggttgcaacagaggtagaccagaagtgtattgacactgctcgtctcaacgtcattgccaacaacctccaagaacgtctcgcaattatagccacctccgtcgatggtcctatacttgtccccctcttgcaggcgaattctgattttgagtacgattttacgatgtgtaatccgcccttctacgatggggcatccgacatgcagacatcggatgctgcgaaggggtttggattcggtgtgaacgctccgcataccggcacggtgcttgagatggccaccgagggaggtgaatcggccttcgtagcccaaatggtccgcgaaagtttgaatcttcaaacacgatgcaggtggttcacgagtaatttggggaaattgaagtccttgtacgaaattgtggggctgctgcgagaacatcagataagtaactacgcaatcaacgaatacgtccaaggagccactcgtcgatatgcgattgcatggtcgttcatcgatgttcgactgcctgatcatttgtcccgtccatctaaccccgacctaagctctcttttctag

SEQ ID NO: 32 - protein, 439 aa, synthetic construct
MQVIPACNSAAIRSLCPTPESFRNMGWLSVSDAVYSEFIGELATRASNRNYSNEFGLMQPIQEFKAFIESDPVVHQEFIDMFEGIQDSPRNYQELCNMFNDIFRKAPVYGDLGPPVYMIMAKLMNTRAGFSAFTRQRLNLHFKKLFDTWGLFLSSKDSRNVLVADQFDDRHCGWLNERALSAMVKHYNGRAFDEVFLCDKNAPYYGFNSYDDFFNRRFRNRDIDRPVVGGVNNTTLISAACESLSYNVSYDVQSLDTLVFKGETYSLKHLLNNDPFTPQFEHGSILQGFLNVTAYHRWHAPVNGTIVKIINVPGTYFAQAPSTIGDPIPDNDYDPPPYLKSLVYFSNIAARQIMFIEADNKEIGLIFLVFIGMTEISTCEATVSEGQHVNRGDDLGMFHFGGSSFALGLRKDCRAEIVEKFTEPGTVIRINEVVAALKA

SEQ ID NO: 33 - DNA, 1320 bp, synthetic construct
  source 1..1320: mol_type = other DNA
atgcaggtgatacccgcgtgcaactcggcagcaataagatcactatgtcctactcccgagtcttttagaaacatgggatggctctctgtcagcgatgcggtctacagcgagttcataggagagttggctacccgcgcttccaatcgaaattactccaacgagttcggcctcatgcaacctatccaggaattcaaggctttcattgaaagcgacccggtggtgcaccaagaatttattgacatgttcgagggcattcaggactctccaaggaattatcaggaactatgtaatatgttcaacgatatctttcgcaaagctcccgtctacggagaccttggccctcccgtttatatgattatggccaaattaatgaacacccgagcgggcttctctgcattcacgagacaaaggttgaaccttcacttcaaaaaacttttcgatacctggggattgttcctgtcttcgaaagattctcgaaatgttcttgtggccgaccagttcgacgacagacattgcggctggttgaacgagcgggccttgtctgctatggttaaacattacaatggacgcgcatttgatgaagtcttcctctgcgataaaaatgccccatactacggcttcaactcttacgacgacttctttaatcgcagatttcgaaaccgagatatcgaccgacctgtagtcggtggagttaacaacaccaccctcatttctgctgcttgcgaatcactttcctacaacgtctcttatgacgtccagtctctcgacactttagttttcaaaggagagacttattcgcttaagcatttgctgaataatgaccctttcaccccacaattcgagcatgggagtattctacaaggattcttgaacgtcaccgcttaccaccgatggcacgcacccgtcaatgggacaatcgtcaaaatcatcaacgttccaggtacctactttgcgcaagccccgagcacgattggcgaccctatcccggataacgattacgacccacctccttaccttaagtctcttgtctacttctctaatattgccgcaaggcaaattatgtttattgaagccgacaacaaggaaattggcctcattttccttgtgttcatcggcatgaccgaaatctcgacatgtgaagccacggtgtccgaaggtcaacacgtcaatcgtggcgatgacttgggaatgttccatttcggtggttcttcgttcgcgcttggtctgaggaaggattgcagggcagagatcgttgaaaagttcaccgaacccggaacagtgatcagaatcaacgaagtcgtcgctgctctaaaggcttag

SEQ ID NO: 34 - protein, 362 aa, synthetic construct
MAFDLKTEDGLITYLTKHLSLDVDTSGVKRLSGGFVNVTWRIKLNAPYQGHTSIILKHAQPHMSTDEDFKIGVERSVYEYQAIKLMMANREVLGGVDGIVSVPEGLNYDLENNALIMQDVGKMKTLLDYVTAKPPLATDIARLVGTEIGGFVARLHNIGRERRDDPEFKFFSGNIVGRTTSDQLYQTIIPNAAKYGVDDPLLPTVVKDLVDDVMHSEETLVMADLWSGNILLQLEEGNPSKLQKIYILDWELCKYGPASLDLGYFLGDCYLISRFQDEQVGTTMRQAYLQSYARTSKHSINYAKVTAGIAAHIVMWTDFMQWGSEEERINFVKKGVAAFHDARGNNDNGEITSTLLKESSTA

SEQ ID NO: 35 - DNA, 1089 bp, synthetic construct
  source 1..1089: mol_type = other DNA
atggcgttcgatctcaagactgaagacggcctcatcacatatctcactaaacatctttctttggacgtcgacacgagcggagtgaagcgccttagcggaggctttgtcaatgtaacctggcgcattaagctcaatgctccttatcaaggtcatacgagcatcatcctgaagcatgctcagccgcacatgtctacggatgaggattttaagataggtgtagaacgttcggtttacgaataccaggctatcaagctcatgatggccaatcgggaggttctgggaggcgtggatggcatagtttctgtgccagaaggcctgaactacgacttagagaataatgcattgatcatgcaagatgtcgggaagatgaagacccttttagattatgtcaccgccaaaccgccacttgcgacggatatagcccgccttgttgggacagaaattggggggttcgttgccagactccataacataggccgcgagaggcgagacgatcctgagttcaaattcttctctggaaatattgtcggaaggacgacttcagaccagctgtatcaaaccatcatacccaacgcagcgaaatatggcgtcgatgaccccttgctgcctactgtggttaaggaccttgtggacgatgtcatgcacagcgaagagacccttgtcatggcggacctgtggagtggaaatattcttctccagttggaggagggaaacccatcgaagctgcagaagatatatatcctggattgggaactttgcaagtacggcccagcgtcgttggacctgggctatttcttgggtgactgctatttgatatcccgctttcaagacgagcaggtcggtacgacgatgcggcaagcctacttgcaaagctatgcgcgtacgagcaagcattcgatcaactacgccaaagtcactgcaggtattgctgctcatattgtgatgtggaccgactttatgcagtgggggagcgaggaagaaaggataaattttgtgaaaaagggggtagctgcctttcacgacgccaggggcaacaacgacaatggggaaattacgtctaccttactgaaggaatcatccactgcgtaa

SEQ ID NO: 36 - protein, 309 aa, synthetic construct
MHIRNPYRTPIDYQALSEAFPPLKPFVSVNADGTSSVDLTIPEAQRAFTAALLHRDFGLTMTIPEDRLCPTVPNRLNYVLWIEDIFNYTNKTLGLSDDRPIKGVDIGTGASAIYPMLACARFKAWSMVGTEVERKCIDTARLNVVANNLQDRLSILETSIDGPILVPIFEATEEYEYEFTMCNPPFYDGAADMQTSDAAKGFGFGVGAPHSGTVIEMSTEGGESAFVAQMVRESLKLRTRCRWYTSNLGKLKSLKEIVGLLKELEISNYAINEYVQGSTRRYAVAWSFTDIQLPEELSRPSNPELSSLF

SEQ ID NO: 37 - DNA, 930 bp, synthetic construct
  source 1..930: mol_type = other DNA
atgcatatcagaaatccttaccgtacaccaattgactatcaagcactttcagaggccttccctcccctcaagccatttgtgtctgtcaatgcagatggtaccagttctgttgacctcactatcccagaagcccagagggcgttcacggccgctcttcttcatcgtgacttcgggctcaccatgaccataccagaagaccgtctgtgcccaacagtccccaataggttgaactacgttctgtggattgaagatattttcaactacacgaacaaaaccctcggcctgtcggatgaccgtcctattaaaggcgttgatattggtacaggagcctccgcaatttatcctatgcttgcctgtgctcggttcaaggcatggtctatggttggaacagaggtcgagaggaagtgcattgacacggcccgcctcaatgtcgtcgcgaacaatctccaagaccgtctctcgatattagagacatccattgatggtcctattctcgtccccattttcgaggcgactgaagaatacgaatacgagtttactatgtgtaaccctccattctacgacggtgctgccgatatgcagacttcggatgctgccaaaggatttggatttggcgtgggcgctccccattctggaacagtcatcgaaatgtcgactgagggaggtgaatcggctttcgtcgctcagatggtccgtgagagcttgaagcttcgaacacgatgcagatggtacacgagtaacttgggaaagctgaaatccttgaaagaaatagtggggctgctgaaagaacttgagataagcaactatgccattaacgaatacgttcaggggtccacacgtcgttatgccgttgcgtggtctttcactgatattcaactgcctgaggagctttctcgtccctctaaccccgagctcagctctcttttctag

SEQ ID NO: 38 - protein, 436 aa, synthetic construct
MQVLTACYTSTLKSLLPSFDAFRSMGWLPVSDKTYNEWIGDLRSRASDKNYTSQVGLIQPIKDFKAFIESDPVVHQEFITMFEGIEESPRNYEELCHMFNDIFRKAPVYGDLGPPVYMVMARIMNTQAGFSAFTKQSLNSHFKRLFDTWGVFLSSKESRYVLVTDQFDDNHYGWLSDRAKSAMVKHYYGRTFEQVFICDEHAPYHGFQSYDDFFNRRFRDRDIDRPVVGGIENTTLISAACESLSYNVCHDLQSLDTLFVKGESYSLKHLLNDDPFARQFEHGSILQGFLNVTAYHRWHAPVNGTILKIINVPGTYFAQAPHTIGDSLDSDHPPYLKSLAYFSNIAARQIMFIEADNKDIGLIFLVFIGMTEISTCEATVSEGQHVNRGDDLGMFHFGGSSFALGLRKDCKAEIFERFAEQGTVIKINEVVAAVKD

SEQ ID NO: 39 - DNA, 1311 bp, synthetic construct
  source 1..1311: mol_type = other DNA
atgcaggtactgaccgcgtgctacacttccacgcttaaatctttactcccaagttttgatgcctttcgaagcatgggatggctgcccgtcagcgacaagacatacaacgaatggataggcgacttgaggagccgcgcatccgacaaaaactacaccagtcaggttggcctcatacagcccatcaaggactttaaagctttcatcgaaagcgaccccgtcgtccatcaagaatttatcacgatgttcgagggcatcgaggagtctccgaggaattatgaggagctatgtcacatgttcaacgatatctttcgcaaagctcccgtctacggagatctaggacccccggtttacatggtcatggccagaataatgaacacacaggctggtttctctgcgttcacaaaacagagtctgaattcccacttcaaacggctcttcgacacttggggtgttttcctttcctcgaaagagtctcgctacgttctcgtgaccgaccagtttgacgacaatcattacggctggctgagcgaccgagccaaatccgccatggtaaaacattactatggtcgcacgttcgaacaggtattcatttgcgacgagcacgcgccataccatggtttccagtcatacgacgactttttcaatcgcagattcagggacagggatattgatcggcctgtcgttggcggcatcgaaaacaccaccctcatttctgccgcatgcgaatctctttcctacaacgtctgccacgatttacaatcactcgacacactattcgtcaaaggcgaatcttattcgctcaagcacttgctcaacgacgacccattcgcacggcaattcgaacacgggagcattcttcagggattcctaaacgttaccgcctaccatcgatggcacgcccccgtcaatggaaccatcctcaaaattatcaacgttcccggtacatactttgcgcaagctcctcacactatcggcgattcgttagacagcgaccaccctccttacctcaagtctcttgcgtacttctccaacatcgccgccaggcaaatcatgtttatcgaagctgacaataaggatatcggccttatcttccttgtctttatcgggatgaccgaaatctccacctgcgaggcgaccgtatctgagggccagcatgtcaatcgaggtgatgatttgggcatgttccacttcgggggttcatcattcgcgcttggtttacgcaaggactgcaaggcggagatttttgaaaggttcgccgaacaaggcactgtcatcaaaattaacgaggttgttgcggctgtcaaagattaa

SEQ ID NO: 40 - protein, 362 aa, synthetic construct
MAFDLKTVEGLIVYLTKCLSLEVDSSGVKRLSGGFVNVTWRIRLNAPYQGHTSIILKHAQPHMSTDKDFKIGVERSVYEYQALKVISANREALGGIDSRVSAPEGLHYDVENNALIMQDVGTLKTLMDYVIEKPAISTEMARLIGTEIGDFVARLHSIGRQKRDQPDFKFFSGNIVGRTTADQLYQTILPNTAKYGIDDPLLPTVVKDLVDEAMQSEETLIMADLWTGNILVEFEEGNLSVLKKIWLVDWELCKYGPVRLDMGYFLGDCFLISRFKNEQVAKAMRQAFLQRYNRVSDTPINYSVATTGIAAHIVMWTDFMNWGTEEERKEYVKKGVAGIHDGRNHNVDGEITSILMQEASTA

SEQ ID NO: 41 - DNA, 1089 bp, synthetic construct
  source 1..1089: mol_type = other DNA
atggctttcgatctcaagactgtagagggcctcatcgtctatcttactaaatgcctgtctttggaggtcgattcgagtggcgtgaagcgcctcagcgggggcttcgtaaatgtaacctggcgcatcaggctcaacgctccttatcagggtcacacgagcatcatcttgaagcatgctcaaccacatatgtcgaccgacaaagattttaagatcggcgtagagcgctcggtgtacgagtatcaggccctcaaggtcatatcagccaatcgagaggccctaggtggtatcgatagccgagtatccgcaccagagggccttcactacgatgtggagaacaatgccctcatcatgcaagatgttgggacgttgaagacgctcatggattatgtcatagaaaaaccggcaatttcgacggagatggcccgtcttatcggtactgagatcggggatttcgtcgccagactccatagcataggccgccaaaagagagatcaacctgatttcaagtttttctctggaaatattgtcgggaggacaactgcagatcaactttatcagactattctacccaacacggcaaaatatggcattgacgaccctcttctccccactgtggtgaaagacctggttgatgaagccatgcagagcgaagaaacacttattatggcagatctgtggactggaaacattctcgtggaattcgaggaaggtaatctatcggtattgaagaagatatggctcgtggactgggagttgtgcaagtatgggcccgtgaggttggatatggggtatttcttgggcgattgtttcttgatctctcgattcaagaacgagcaagtcgcaaaggcaatgcgacaagctttcctgcaacgttataatcgagtttctgatacaccgatcaactactccgttgcgacgactggcatcgctgcccacatcgttatgtggactgactttatgaactggggcacagaagaggaaaggaaagagtacgtgaagaaaggtgtcgcaggaatccatgacgggcgaaaccacaacgtagatggggagattacgtccattctaatgcaggaagcatcgacggcgtag

SEQ ID NO: 42 - protein, 317 aa, synthetic construct
MHNRNPYRDVIDYQALAEAYPPLKPHVTVNADNTASIDLTIPEVQRQYTAALLHRDFGLTITLPEDRLCPTVPNRLNYVLWIEDIFQCTNKALGLSDDRPVKGVDIGTGASAIYPMLACARFKQWSMIATEVERKCIDTARLNVLANNLQDRLSILEVSVDGPILVPIFDTFERATSDYEFEFTMCNPPFYDGAADMQTSDAAKGFGFGVNAPHSGTVIEMATEGGEAAFVAQMVRESMKLQTRCRWFTSNLGKLKSLHEIVALLRESQITNYAINEYVQGTTRRYALAWSFTDIKLTEELYRPSNPELGPLCSTFV

SEQ ID NO: 43 - DNA, 954 bp, synthetic construct
  source 1..954: mol_type = other DNA
atgcacaacagaaacccataccgcgatgttatcgactaccaagctctggctgaggcgtatccgcccctcaagccacatgtgactgtcaatgctgacaatacggcatccatcgacctcaccatcccagaagtgcaaaggcaatatacagctgcacttcttcatcgtgacttcggtctgacgattacactcccagaagaccgtctttgcccaacagtgccaaacaggctgaactatgtcctttggattgaggacatcttccagtgcactaataaggctcttggtctctcagatgaccgtcctgtcaaaggcgttgacataggaactggtgcctcagcaatctatcctatgctggcctgtgcgcgtttcaagcaatggtccatgattgcaacagaggtcgaacgcaaatgtattgacacggcccgtttgaacgtcttggccaacaatctccaagaccgtctctctatcttggaggtttccgtcgatggtcctatccttgttcccatcttcgacactttcgaaagggcaacctcggactacgagttcgagttcacgatgtgtaacccccctttctacgatggtgcagctgacatgcaaacttccgatgccgcaaaaggctttggatttggggtgaatgcgccacattccggaactgtgatcgaaatggccactgagggaggtgaagcggcctttgtcgcccaaatggttcgtgaaagcatgaaacttcaaacacgatgcagatggttcacgagcaacttgggaaagttgaagtccttgcatgagatagtggctctcctgagggaatctcagatcactaactacgcaatcaatgagtatgtccaagggaccactcgtcgctacgctcttgcttggtcttttaccgatattaaattgactgaggaattgtaccgcccatctaaccctgaattgggtcctctttgctcgacctttgtctga

(19) United States
(12) Patent Application Publication
(10) Pub. No.: US 2026/0159865 A1
(43) Pub. Date: Jun. 11, 2026

(54) METHODS FOR THE IMPROVED PRODUCTION OF PSILOCYBIN AND INTERMEDIATES OR SIDE PRODUCTS THROUGH ENZYME OPTIMIZATION

(71) Applicant: Miami University, Oxford, OH (US)

(72) Inventors: John Andrew Jones, Liberty Township, OH (US); Madeline McKinney, Morrow, OH (US)

(21) Appl. No.: 18/706,856

(22) PCT Filed: Nov. 4, 2022
(86) PCT No.: PCT/US2022/079308

§ 371 Date: May 2, 2024

Related U.S. Application Data
(60) Provisional application No. 63/263,607, filed on Nov. 5, 2021.

(51) Int. Cl. C12P 17/10; C12N 1/20; C12N 9/10; C12N 9/12; C12N 9/88; C12N 15/70; C12R 1/19

(52) U.S. Cl. CPC C12P 17/10; C12N 1/20; C12N 9/1007; C12N 9/1205; C12N 9/88; C12N 15/70; C12R 2001/19; C12Y 201/01; C12Y 207/01; C12Y 401/01

(57) ABSTRACT

Provided are methods, prokaryotic host cells, expression vectors, and kits for the production of psilocybin or an intermediate or a side product thereof using at least one psilocybin production gene from Psilocybe cyanescens, Panaeolus cyanescens, Gymnopilus dilepis, or Gymnopilus junonius. Also provided are methods, prokaryotic host cells, expression vectors, and kits for the production of norbaeocystin using at least one psilocybin production gene from Psilocybe cyanescens, Panaeolus cyanescens, Gymnopilus dilepis, or Gymnopilus junonius. In certain embodiments, the prokaryotic host cell is selected from the group consisting of Escherichia coli, Corynebacterium glutamicum, Vibrio natriegens, Bacillus subtilis, Bacillus megaterium, Escherichia coli Nissle 1917, Clostridium acetobutlyicum, Streptomyces coelicolor, Lactococcus lactis, Pseudomonas putida, Streptomyces clavuligerus, and Streptomyces venezuelae.

### CROSS-REFERENCE TO RELATED APPLICATIONS

The instant application is entitled to priority under 35 U.S.C. § 119(e) to U.S. Provisional Application No. 63/263,607 filed Nov. 5, 2021, which is hereby incorporated by reference in its entirety.

The general inventive concepts relate to the field of medical therapeutics and more particularly to improved methods for the production of psilocybin and intermediates or side products through enzyme optimization.

### SEQUENCE LISTING

The contents of the electronic sequence listing (315691-00042.xml; Size: 57,552 bytes, and Date of Creation: Nov. 4, 2022) is herein incorporated by reference in its entirety.

### BACKGROUND

Approximately 1 out of 5 adults are currently living with some type of mental illness¹, and current standards of care come with a plethora of side effects, including weight gain, headaches, and anxiety². Psilocybin (4-phosphoryloxy-N,N-dimethyltryptamine), the active ingredient in “magic mushrooms.” is currently under clinical evaluation for the treatment of severe depression³, post-traumatic stress disorder (PTSD)⁴, and anxiety⁵. Additionally, anecdotal evidence from recreational users has led some to postulate that the ratio of naturally occurring psychoactive metabolites in various mushroom species may greatly impact the psychedelic experience and overall effect on the brain⁶. Notably, the consumption of Inocybe aeruginascens, a species containing notable quantities of baeocystin, psilocybin, and aeruginascin, frequently elicits a more euphoric hallucination experience as compared with that of the more common recreationally used species, Psilocybe cubensis⁷. This “Entourage Effect” as it is known, stands on the premise that different ratios of norbaeocystin, baeocystin, psilocybin, and aeruginascin can significantly influence the constructive impact on the brain.

Much of the interest in psilocybin is due to its biosynthetic precursors-norbaeocystin and baeocystin. These compounds have structural similarity to the neurotransmitter serotonin and sparked the interest of researchers who were curious to understand the mechanism behind their hallucinogenic properties. Clinical trials with psilocybin as a medication for individuals struggling with treatment-resistant depression are ongoing.

There remains a need for methods for the improved production of psilocybin and intermediates or side products thereof.

### SUMMARY

The general inventive concepts relate to and contemplate methods and compositions for producing psilocybin or an intermediate or a side product thereof.

Provided is a method for the production of psilocybin or an intermediate or a side product thereof comprising contacting a prokaryotic host cell with one or more expression vectors, wherein each expression vector comprises a psilocybin production gene selected from the group consisting of psiD, psiK and psiM and combinations thereof; and culturing the host cell; wherein at least one psilocybin production gene is from Psilocybe cyanescens, Panaeolus cyanescens, Gymnopilus dilepis, or Gymnopilus junonius. In some embodiments, at least one expression vector further comprises a psilocybin production gene selected from the group consisting of psiD, psiK and psiM and combinations thereof from Psilocybe cubensis. In some embodiments, the psilocybin production gene from Psilocybe cubensis is selected from the group consisting of psiD and psiK and combinations thereof.

In some embodiments, the prokaryotic host cell is further contacted with at least one expression vector comprising a psilocybin production gene selected from the group consisting of psiD, psiK and psiM and combinations thereof from Psilocybe cubensis. In some embodiments, the psilocybin production gene from Psilocybe cubensis is selected from the group consisting of psiD and psiK and combinations thereof.

In certain embodiments, the prokaryotic host cell is selected from the group consisting of Escherichia coli, Corynebacterium glutamicum, Vibrio natriegens, Bacillus subtilis, Bacillus megaterium, Escherichia coli Nissle 1917, Clostridium acetobutlyicum, Streptomyces coelicolor, Lactococcus lactis, Pseudomonas putida, Streptomyces clavuligerus, and Streptomyces venezuelae.

In some embodiments, the intermediate or side product of psilocybin is norbaeocystin, baeocystin, 4-hydroxytryptophan, 4-hydroxytryptamine, aeruginascin, psilocin, norpsilocin, or 4-hydroxy-N,N,N-trimethyltryptamine (4-OH-TMT). In some embodiments the intermediate of psilocybin is norbaeocystin, baeocystin, 4-hydroxytryptophan, or 4-hydroxytryptamine. In some embodiments, the side product of psilocybin is aeruginascin, psilocin, norpsilocin, or 4-hydroxy-N,N,N-trimethyltryptamine (4-OH-TMT).

Also provided is a recombinant prokaryotic cell comprising one or more expression vectors, wherein each expression vector comprises a psilocybin production gene selected from the group consisting of psiD, psiK and psiM and combinations thereof; wherein at least one psilocybin production gene is from Psilocybe cyanescens. Panaeolus cyanescens. Gymnopilus dilepis, or Gymnopilus junonius. In some embodiments, at least one expression vector further comprises a psilocybin production gene selected from the group consisting of psiD, psiK and psiM and combinations thereof from Psilocybe cubensis. In some embodiments, the psilocybin production gene from Psilocybe cubensis is selected from the group consisting of psiD and psiK and combinations thereof.

In some embodiments, the prokaryotic host cell further comprises at least one expression vector comprising a psilocybin production gene selected from the group consisting of psiD, psiK and psiM and combinations thereof from Psilocybe cubensis. In some embodiments, the psilocybin production gene from Psilocybe cubensis is selected from the group consisting of psiD and psiK and combinations thereof.

Provided is a vector for introducing at least one gene associated with psilocybin production; the gene may be selected from: psiD, psiK, and psiM and combinations thereof; wherein at least one psilocybin production gene is from Psilocybe cyanescens, Panaeolus cyanescens, Gymnopilus dilepis, or Gymnopilus junonius. Also provided is a transfection kit comprising an expression vector as described herein.

Provided is a method for the production of norbaeocystin comprising contacting a prokaryotic host cell with one or more expression vectors, wherein each expression vector comprises a psilocybin production gene selected from the group consisting of psiD and psiK and combinations thereof; and culturing the host cell; wherein at least one psilocybin production gene is from Psilocybe cyanescens, Panaeolus cyanescens, Gymnopilus dilepis, or Gymnopilus junonius. In some embodiments, at least one expression vector further comprises a psilocybin production gene selected from the group consisting of psiD and psiK and combinations thereof from Psilocybe cubensis. In some embodiments, the prokaryotic host cell is further contacted with at least one expression vector comprising a psilocybin production gene selected from the group consisting of psiD and psiK and combinations thereof from Psilocybe cubensis.

In certain embodiments, none of the expression vectors comprises psiM.

In certain embodiments, the prokaryotic host cell is selected from the group consisting of Escherichia coli, Corynebacterium glutamicum, Vibrio natriegens, Bacillus subtilis, Bacillus megaterium, Escherichia coli Nissle 1917, Clostridium acetobutlyicum, Streptomyces coelicolor, Lactococcus lactis, Pseudomonas putida, Streptomyces clavuligerus, and Streptomyces venezuelae.

Also provided is a recombinant prokaryotic cell comprising one or more expression vectors, wherein each expression vector comprises a psilocybin production gene selected from the group consisting of psiD, psiK, and combinations thereof; wherein at least one psilocybin production gene is from Psilocybe cyanescens, Panaeolus cyanescens, Gymnopilus dilepis, or Gymnopilus junonius. In some embodiments, at least one expression vector further comprises a psilocybin production gene selected from the group consisting of psiD, psiK, and combinations thereof from Psilocybe cubensis. In some embodiments, the prokaryotic host cell further comprises at least one expression vector comprising a psilocybin production gene selected from the group consisting of psiD, psiK, and combinations thereof from Psilocybe cubensis.

Provided is a vector for introducing at least one gene associated with psilocybin production; the gene may be selected from: psD, psiK, and combinations thereof; wherein at least one psilocybin production gene is from Psilocybe cyanescens, Panaeolus cyanescens, Gymnopilus dilepis, or Gymnopilus junonius. In some embodiments, at least one psilocybin production gene is from Psilocybe cubensis. Also provided is a transfection kit comprising an expression vector as described herein.

### DESCRIPTION OF THE FIGURES

FIG. 1 illustrates a Psilocybin Biosynthetic Pathway. As aeruginascin showed no significant accumulation, the final methylation performed by psiM is crossed out below.

FIG. 2 shows the sequence alignment of 4 norbaeocystin methyltransferases (PsiM) with highly conserved regions highlighted. Gymnopilus dilepis denoted as gymdi (SEQ ID NO:22), Psilocybe cyanescens as psicy (SEQ ID NO:30), Psilocybe cubensis denoted as psicu (SEQ ID NO:36), and Panaeolus cyanescens as pancy (SEQ ID NO:42).

FIGS. 3A-3D show preliminary screening and selection of strains of interest. Psilocybin and baeocystin production from (FIG. 3A) Psilocybe cubensis PsiM library, (FIG. 3B) Gymnopilus dilepis PsiM library, (FIG. 3C) Psilocybe cyanescens PsiM library, and (FIG. 3D) Panaeolus cyanescens PsiM library. Strains chosen for further experimentation are denoted with a black star.

FIG. 4 illustrates selected mutant validation. Psilocybe cubensis denoted as psicu, Gymnopilus dilepis as gymdi, Psilocybe cyanescens as psicy, and Panaeolus cyanescens as pancy.

FIG. 5 shows production of psilocybin and baeocystin as a function of time. Left panel: Pancy 10. Right panel: Gymdi30. Error bars represent one standard deviation of the duplicates (N=2).

FIG. 6 illustrates operon configuration. Black diamonds represent ribosome binding sites, the black “T” represents the terminator, and the light gray arrow represents one of 7 possible promoters. Both psiD and psiK genes are from Psilocybe cubensis while the psiM arrow has an X to denote the various species under investigation.

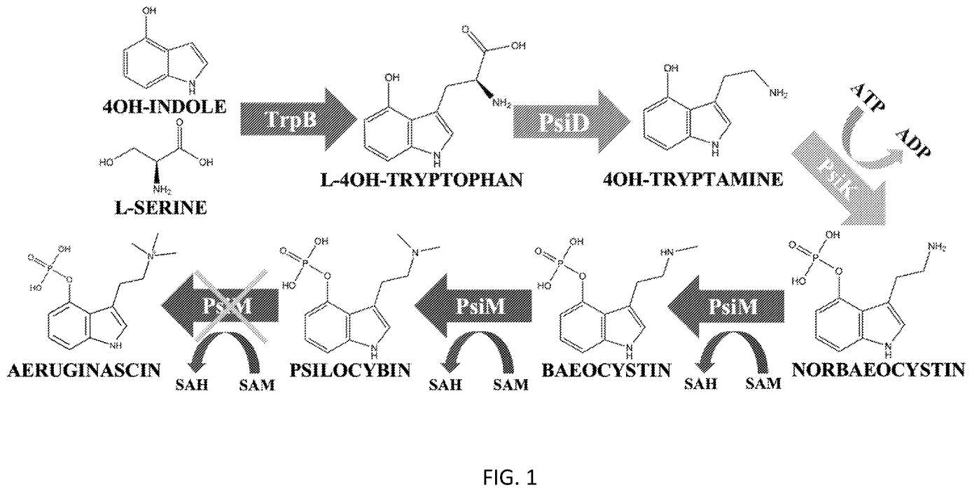

### DETAILED DESCRIPTION

While the general inventive concepts are susceptible of embodiment in many forms, there are shown in the drawings, and will be described herein in detail, specific embodiments thereof with the understanding that the present disclosure is to be considered an exemplification of the principles of the general inventive concepts. Accordingly, the general inventive concepts are not intended to be limited to the specific embodiments illustrated herein.

It is also to be understood that the terminology used herein is for the purpose of describing particular embodiments only, and is not intended to be limiting.

The articles “a” and “an” are used herein to refer to one or more than one (i.e., to at least one) of the grammatical object of the article. By way of example, “a cell” means one cell or more than one cell.

“About” as used herein when referring to a measurable value such as an amount, a temporal duration, and the like, is meant to encompass variations of ±5%, preferably ±1%, and still more preferably ±0.1% from the specified value, as such variations are appropriate to perform the disclosed methods.

Embodiments described herein as “comprising” one or more features may also be considered as disclosure of the corresponding embodiments “consisting of” and/or “consisting essentially of” such features, and vice-versa.

Concentrations, amounts, volumes, percentages and other numerical values may be presented herein in a range format. It is also to be understood that such range format is used merely for convenience and brevity and should be interpreted flexibly to include not only the numerical values explicitly recited as the limits of the range but also to include all the individual numerical values or sub-ranges encompassed within that range as if each numerical value and sub-range in explicitly recited.

As used herein, the term “prokaryotic host cell” means a prokaryotic cell that is susceptible to transformation, transfection, transduction, or the like, with a nucleic acid construct or expression vector comprising a polynucleotide. The term “prokaryotic host cell” encompasses any progeny that is not identical due to mutations that occur during replication.

As used herein, the term “recombinant cell” or “recombinant host” means a cell or host cell that has been genetically modified or altered to comprise a nucleic acid sequence that is not native to the cell or host cell. In some embodiments the genetic modification comprises integrating the polynucleotide in the genome of the host cell. In further embodiments the polynucleotide is exogenous in the host cell.

As used herein, the term “intermediate” of psilocybin means an intermediate in the production or biosynthesis of psilocybin, e.g., norbacocystin, bacocystin, 4-hydroxytryptophan, 4-hydroxytryptamine.

As used herein, the term “side product” of psilocybin means a side product in the production or biosynthesis of psilocybin, e.g., aeruginascin, psilocin, norpsilocin, or 4-hydroxy-N,N,N-trimethyltryptamine (4-OH-TMT).

The materials, compositions, and methods described herein are intended to be used to provide novel routes for the production of psilocybin and intermediates or side products, and methods for the production of norbaeocystin.

### I. Methods, Vectors, Host Cells and Kits for the Production of Psilocybin or an Intermediate or a Side Product Thereof

### Methods

Provided is a method for the production of psilocybin or an intermediate or a side product thereof. The method comprises contacting a host cell with at least one psilocybin production gene selected from: psiD, psiK, psiM, and combinations thereof to form a recombinant cell; culturing the recombinant cell; and obtaining the psilocybin; wherein at least one psilocybin production gene is from Psilocybe cyanescens, Panaeolus cyanescens, Gymnopilus dilepis, or Gymnopilus junonius. In some embodiments, at least one expression vector further comprises a psilocybin production gene selected from the group consisting of psiD, psiK and psiM and combinations thereof from Psilocybe cubensis. In some embodiments, the psilocybin production gene from Psilocybe cubensis is selected from the group consisting of psiD and psiK and combinations thereof.

In some embodiments, the prokaryotic host cell is further contacted with at least one expression vector comprising a psilocybin production gene selected from the group consisting of psiD, psiK and psiM and combinations thereof from Psilocybe cubensis. In some embodiments, the psilocybin production gene from Psilocybe cubensis is selected from the group consisting of psiD and psiK and combinations thereof.

In certain embodiments, the host cell is a prokaryotic cell. In certain exemplary embodiments, the host cell is an E. coli cell.

Provided is a method for the production of psilocybin or an intermediate or a side product thereof comprising contacting a prokaryotic host cell with one or more expression vectors, wherein each expression vector comprises a psilocybin production gene selected from the group consisting of psiD, psiK and psiM and combinations thereof; and culturing the host cell: wherein at least one psilocybin production gene is from Psilocybe cyanescens, Panaeolus cyanescens, Gymnopilus dilepis, or Gymnopilus junonius. In some embodiments, at least one expression vector further comprises a psilocybin production gene selected from the group consisting of psiD, psiK and psiM and combinations thereof from Psilocybe cubensis. In some embodiments, the psilocybin production gene from Psilocybe cubensis is selected from the group consisting of psiD and psiK and combinations thereof.

In some embodiments, the prokaryotic host cell is further contacted with at least one expression vector comprising a psilocybin production gene selected from the group consisting of psiD, psiK and psiM and combinations thereof from Psilocybe cubensis. In some embodiments, the psilocybin production gene from Psilocybe cubensis is selected from the group consisting of psiD and psiK and combinations thereof.

In certain embodiments, the prokaryotic host cell is selected from the group consisting of Escherichia coli, Corynebacterium glutamicum, Vibrio natriegens, Bacillus subtilis, Bacillus megaterium, Escherichia coli Nissle 1917, Clostridium acetobutlyicum, Streptomyces coelicolor, Lactococcus lactis, Pseudomonas putida, Streptomyces clavuligerus, and Streptomyces venezuelae.

In certain embodiments, the psiD gene encodes a polypeptide comprising the amino acid sequence of SEQ ID NO: 18, 26, 32, or 38, or a sequence having at least 60%, at least 70%, at least 80%, at least 90%, at least 95%, at least 96%, at least 97%, at least 98%, or at least 99% sequence identity thereto. In certain embodiments, the psiD comprises the amino acid sequence of Genbank accession number PPQ70875, KY984104, KY984101.1, PPQ80975, or a sequence having at least 60%, at least 70%, at least 80%, at least 90%, at least 95%, at least 96%, at least 97%, at least 98%, or at least 99% sequence identity thereto. In certain embodiments, the psiD is encoded by a nucleotide sequence comprising SEQ ID NO: 19, 27, 33, or 39, or a sequence having at least 60%, at least 70%, at least 80%, at least 90%, at least 95%, at least 96%, at least 97%, at least 98%, or at least 99% sequence identity thereto.

In certain embodiments, the psiK gene encodes a polypeptide comprising the amino acid sequence of SEQ ID NO: 20, 28, 34, or 40, or a sequence having at least 60%, at least 70%, at least 80%, at least 90%, at least 95%, at least 96%, at least 97%, at least 98%, or at least 99% sequence identity thereto. In certain embodiments, the psiK comprises the amino acid sequence of Genbank accession number PPQ70874, KY984102, KY984099.1, PPQ98758, or a sequence having at least 60%, at least 70%, at least 80%, at least 90%, at least 95%, at least 96%, at least 97%, at least 98%, or at least 99% sequence identity thereto. In certain embodiments, the psiK is encoded by a nucleotide sequence comprising SEQ ID NO: 21, 29, 35, or 41, or a sequence having at least 60%, at least 70%, at least 80%, at least 90%, at least 95%, at least 96%, at least 97%, at least 98%, or at least 99% sequence identity thereto.

In certain embodiments, the psiM gene encodes a polypeptide comprising the amino acid sequence of SEQ ID NO: 22, 24, 30, 36, or 42, or a sequence having at least 60%, at least 70%, at least 80%, at least 90%, at least 95%, at least 96%, at least 97%, at least 98%, or at least 99% sequence identity thereto. In certain embodiments, the psiM comprises the amino acid sequence of Genbank accession number PPQ70884, KY984103, KY984100.1, PPQ80976, or a sequence having at least 60%, at least 70%, at least 80%, at least 90%, at least 95%, at least 96%, at least 97%, at least 98%, or at least 99% sequence identity thereto. In certain embodiments, the psiM is encoded by a nucleotide sequence comprising SEQ ID NO: 23, 25, 31, 37, or 43, or a sequence having at least 60%, at least 70%, at least 80%, at least 90%, at least 95%, at least 96%, at least 97%, at least 98%, or at least 99% sequence identity thereto.

In certain embodiments, the prokaryotic cell is contacted with an expression vector comprising a psiD gene, a psiK gene and a psiM gene all under control of a single promoter in operon configuration; wherein at least one gene is from Psilocybe cyanescens, Panaeolus cyanescens, Gymnopilus dilepis, or Gymnopilus junonius. In some embodiments, at least one psilocybin production gene is from Psilocybe cubensis. In certain embodiments, the prokaryotic cell is contacted with an expression vector comprising a psiD gene, a psiK gene and a psiM gene, wherein each gene is under control of a separate promoter in pseudooperon configuration: wherein at least one gene is from Psilocybe cyanescens, Panaeolus cyanescens, Gymnopilus dilepis, or Gymnopilus junonius. In some embodiments, at least one psilocybin production gene is from Psilocybe cubensis. In certain embodiments, each gene is in monocistronic configuration, wherein each gene has a promoter and a terminator. Any configuration or arrangement of promoters and terminators is envisaged.

In some embodiments, the promoter is selected from the group consisting of G6 mutant T7, H9 mutant T7, H10 mutant T7, C4 mutant 17, consensus T7, Lac, Lac UV5, tac, trc, GAP, and xylA promoter.

It is envisaged that any intermediate or side product of psilocybin may be produced by any of the methods described herein. In some embodiments, the intermediate or side product of psilocybin is norbaeocystin, baeocystin, 4-hydroxytryptophan, 4-hydroxytryptamine, aeruginascin, psilocin, norpsilocin, or 4-hydroxy-N,N,N-trimethyltryptamine (4-OH-TMT). In some embodiments the intermediate of psilocybin is norbaeocystin, baeocystin, 4-hydroxytryptophan, or 4-hydroxytryptamine. In some embodiments, the side product of psilocybin is aeruginascin, psilocin, norpsilocin, or 4-hydroxy-N,N,N-trimethyltryptamine (4-OH-TMT).

In certain embodiments, the host cell is cultured with a supplement independently selected from the group consisting of 4-hydroxyindole, serine, methionine, 4-hydroxytryptophan, 4-hydroxytryptamine, and combinations thereof. In certain exemplary embodiments, the supplement is fed continuously to the host cell. In further embodiments, the host cell is grown in an actively growing culture. Continuous feeding is accomplished by using a series of syringe and/or peristaltic pumps whose outlet flow is directly connected to the bioreactor. The set point of these supplement addition pumps is adjusted in response to real-time measurement of cell biomass and specific metabolic levels using UV-vis absorption and HPLC analysis, respectively. The fed-batch fermentation process is focused on maximizing production of target metabolites through harnessing the ability of an actively growing and replicating cell culture to regenerate key co-factors and precursors which are critical to the biosynthesis of target metabolites. This process notably does not involve the centrifugal concentration and reconstitution of cell biomass to artificially higher cell density and/or into production media that was not used to build the initial biomass. The production process involves the inoculation of the reactor from an overnight preculture at low optical density, followed by exponential phase growth entering into a fed-batch phase of production, culminating in a high cell density culture.

The psilocybin and intermediate or side products are found extracellularly in the fermentation broth. In certain embodiments, the psilocybin and intermediate or side products are isolated. These target products can be collected through drying the fermentation broth after centrifugation to remove the cell biomass. The resulting dry product can be extracted to further purify the target compounds. Alternatively, the products can be extracted from the liquid cell culture broth using a solvent which is immiscible with water and partitions psilocybin or any of the intermediate or side products into the organic phase. Furthermore, contaminants from the fermentation broth can be removed through extraction leaving the psilocybin and/or intermediate or side products in the aqueous phase for collection after drying or crystallization procedures.

In certain embodiments, the methods described herein result in a titer of psilocybin of about 0.1 to about 50 g/L. In some embodiments, the methods described herein result in a titer of psilocybin of about 0.1 to about 10 g/L. In yet further embodiments, the methods described herein result in a titer of psilocybin of about 0.1 to about 5 g/L. In certain embodiments, the methods described herein result in a titer of psilocybin of about 0.4 to about 3 g/L. In further embodiments, the methods described herein result in a titer of psilocybin of about 0.5 to about 2.5 g/L. In yet further embodiments, the methods described herein result in a titer of psilocybin of about 1.1 g/L.

In certain embodiments, the methods described herein result in a molar yield of psilocybin of about 10% to about 100%. In some embodiments, the methods described herein result in a molar yield of psilocybin of about 20% to about 80%. In yet further embodiments, the methods described herein result in a molar yield of psilocybin of about 30% to about 70%. In certain embodiments, the methods described herein result in a molar yield of psilocybin of about 40% to about 60%. In further embodiments, the methods described herein result in a molar yield of psilocybin of about 50%.

### Recombinant Prokaryotic Cells for the Production of Psilocybin or an Intermediate or a Side Product Thereof

Provided is a recombinant prokaryotic cell comprising one or more expression vectors, wherein each expression vector comprises a psilocybin production gene selected from the group consisting of psiD, psiK and psiM and combinations thereof; wherein at least one psilocybin production gene is from Psilocybe cyanescens, Panaeolus cyanescens, Gymnopilus dilepis, or Gymnopilus junonius. In some embodiments, at least one expression vector further comprises a psilocybin production gene selected from the group consisting of psiD, psiK and psiM and combinations thereof from Psilocybe cubensis. In some embodiments, the psilocybin production gene from Psilocybe cubensis is selected from the group consisting of psiD and psiK and combinations thereof.

In some embodiments, the prokaryotic host cell further comprises at least one expression vector comprising a psilocybin production gene selected from the group consisting of psiD, psiK and psiM and combinations thereof from Psilocybe cubensis. In some embodiments, the psilocybin production gene from Psilocybe cubensis is selected from the group consisting of psiD and psiK and combinations thereof.

In certain embodiments, the recombinant prokaryotic cell is selected from the group consisting of Escherichia coli, Corynebacterium glutamicum, Vibrio natriegens, Bacillus subtilis, Bacillus megaterium, Escherichia coli Nissle 1917, Clostridium acetobutlyicum, Streptomyces coelicolor, Lactococcus lactis, Pseudomonas putida, Streptomyces clavuligerus, and Streptomyces venezuelae.

In certain embodiments, the psiD gene encodes a polypeptide comprising the amino acid sequence of SEQ ID NO: 18, 26, 32, or 38, or a sequence having at least 60%, at least 70%, at least 80%, at least 90%, at least 95%, at least 96%, at least 97%, at least 98%, or at least 99% sequence identity thereto. In certain embodiments, the psiD comprises the amino acid sequence of Genbank accession number PPQ70875, KY984104, KY984101.1, PPQ80975, or a sequence having at least 60%, at least 70%, at least 80%, at least 90%, at least 95%, at least 96%, at least 97%, at least 98%, or at least 99% sequence identity thereto. In certain embodiments, the psiD is encoded by a nucleotide sequence comprising SEQ ID NO: 19, 27, 33, or 39, or a sequence having at least 60%, at least 70%, at least 80%, at least 90%, at least 95%, at least 96%, at least 97%, at least 98%, or at least 99% sequence identity thereto.

In certain embodiments, the psiK gene encodes a polypeptide comprising the amino acid sequence of SEQ ID NO: 20, 28, 34, or 40, or a sequence having at least 60%, at least 70%, at least 80%, at least 90%, at least 95%, at least 96%, at least 97%, at least 98%, or at least 99% sequence identity thereto. In certain embodiments, the psiK comprises the amino acid sequence of Genbank accession number PPQ70874, KY984102, KY984099.1, PPQ98758, or a sequence having at least 60%, at least 70%, at least 80%, at least 90%, at least 95%, at least 96%, at least 97%, at least 98%, or at least 99% sequence identity thereto. In certain embodiments, the psiK is encoded by a nucleotide sequence comprising SEQ ID NO: 21, 29, 35, or 41, or a sequence having at least 60%, at least 70%, at least 80%, at least 90%, at least 95%, at least 96%, at least 97%, at least 98%, or at least 99% sequence identity thereto.

In certain embodiments, the psiM gene encodes a polypeptide comprising the amino acid sequence of SEQ ID NO: 22, 24, 30, 36, or 42, or a sequence having at least 60%, at least 70%, at least 80%, at least 90%, at least 95%, at least 96%, at least 97%, at least 98%, or at least 99% sequence identity thereto. In certain embodiments, the psiM comprises the amino acid sequence of Genbank accession number PPQ70884, KY984103, KY984100.1, PPQ98758, or a sequence having at least 60%, at least 70%, at least 80%, at least 90%, at least 95%, at least 96%, at least 97%, at least 98%, or at least 99% sequence identity thereto. In certain embodiments, the psiM is encoded by a nucleotide sequence comprising SEQ ID NO: 23, 25, 31, 37, or 43, or a sequence having at least 60%, at least 70%, at least 80%, at least 90%, at least 95%, at least 96%, at least 97%, at least 98%, or at least 99% sequence identity thereto.

In certain embodiments, the prokaryotic cell is contacted with an expression vector comprising a psiD gene, a psiK gene and a psiM gene all under control of a single promoter in operon configuration; wherein at least one psilocybin production gene is from Psilocybe cyanescens, Panaeolus cyanescens, Gymnopilus dilepis, or Gymnopilus junonius. In some embodiments, at least one psilocybin production gene is from Psilocybe cubensis. In certain embodiments, the prokaryotic cell is contacted with an expression vector comprising a psiD gene, a psiK gene and a psiM gene, wherein each gene is under control of a separate promoter in pseudooperon configuration; wherein at least one psilocybin production gene is from Psilocybe cyanescens, Panaeolus cyanescens, Gymnopilus dilepis, or Gymnopilus junonius. In some embodiments, at least one psilocybin production gene is from Psilocybe cubensis. In certain embodiments, each gene is in monocistronic configuration, wherein each gene has a promoter and a terminator. Any configuration or arrangement of promoters and terminators is envisaged.

In some embodiments, the promoter is selected from the group consisting of G6 mutant T7, H9 mutant 17, H10 mutant T7, C4 mutant T7, consensus T7, Lac, Lac UV5, tac, trc, GAP, and xylA promoter.

### Expression Vectors

Provided is a vector for introducing at least one gene associated with psilocybin production; the gene may be selected from: psiD, psiK, and psiM and combinations thereof; wherein at least one psilocybin production gene is from Psilocybe cyanescens, Panaeolus cyanescens, Gymnopilus dilepis, or Gymnopilus junonius. In some embodiments, the vector further comprises a psilocybin production gene selected from the group consisting of psiD, psiK and psiM and combinations thereof from Psilocybe cubensis.

In certain embodiments, the psiD gene encodes a polypeptide comprising the amino acid sequence of SEQ ID NO: 18, 26, 32, or 38, or a sequence having at least 60%, at least 70%, at least 80%, at least 90%, at least 95%, at least 96%, at least 97%, at least 98%, or at least 99% sequence identity thereto. In certain embodiments, the psiD comprises the amino acid sequence of Genbank accession number PPQ70875, KY984104, KY984101.1, PPQ80975, or a sequence having at least 60%, at least 70%, at least 80%, at least 90%, at least 95%, at least 96%, at least 97%, at least 98%, or at least 99% sequence identity thereto. In certain embodiments, the psiD is encoded by a nucleotide sequence comprising SEQ ID NO: 19, 27, 33, or 39, or a sequence having at least 60%, at least 70%, at least 80%, at least 90%, at least 95%, at least 96%, at least 97%, at least 98%, or at least 99% sequence identity thereto.

In certain embodiments, the psiK gene encodes a polypeptide comprising the amino acid sequence of SEQ ID NO: 20, 28, 34, or 40, or a sequence having at least 60%, at least 70%, at least 80%, at least 90%, at least 95%, at least 96%, at least 97%, at least 98%, or at least 99% sequence identity thereto. In certain embodiments, the psiK comprises the amino acid sequence of Genbank accession number PPQ70874, KY984102, KY984099.1, PPQ98758, or a sequence having at least 60%, at least 70%, at least 80%, at least 90%, at least 95%, at least 96%, at least 97%, at least 98%, or at least 99% sequence identity thereto. In certain embodiments, the psiK is encoded by a nucleotide sequence comprising SEQ ID NO: 21, 29, 35, or 41, or a sequence having at least 60%, at least 70%, at least 80%, at least 90%, at least 95%, at least 96%, at least 97%, at least 98%, or at least 99% sequence identity thereto.

In certain embodiments, the psiM gene encodes a polypeptide comprising the amino acid sequence of SEQ ID NO: 22, 24, 30, 36, or 42, or a sequence having at least 60%, at least 70%, at least 80%, at least 90%, at least 95%, at least 96%, at least 97%, at least 98%, or at least 99% sequence identity thereto. In certain embodiments, the psiM comprises the amino acid sequence of Genbank accession number PPQ70884, KY984103, KY984100.1, PPQ98758, or a sequence having at least 60%, at least 70%, at least 80%, at least 90%, at least 95%, at least 96%, at least 97%, at least 98%, or at least 99% sequence identity thereto. In certain embodiments, the psiM is encoded by a nucleotide sequence comprising SEQ ID NO: 23, 25, 31, 37, or 43, or a sequence having at least 60%, at least 70%, at least 80%, at least 90%, at least 95%, at least 96%, at least 97%, at least 98%, or at least 99% sequence identity thereto.

In certain embodiments, the expression vector comprises a psiD gene, a psiK gene and a psiM gene all under control of a single promoter in operon configuration; wherein at least one psilocybin production gene is from Psilocybe cyanescens, Panaeolus cyanescens, Gymnopilus dilepis, or Gymnopilus junonius. In some embodiments, at least one psilocybin production gene is from Psilocybe cubensis.

In certain embodiments, the expression vector comprises a psiD gene, a psiK gene and a psiM gene, wherein each gene is under control of a separate promoter in pseudooperon configuration: wherein at least one psilocybin production gene is from Psilocybe cyanescens, Panaeolus cyanescens, Gymnopilus dilepis, or Gymnopilus junonius. In some embodiments, at least one psilocybin production gene is from Psilocybe cubensis.

In certain embodiments, each gene is in monocistronic configuration, wherein each gene has a promoter and a terminator. Any configuration or arrangement of promoters and terminators is envisaged.

In some embodiments, the promoter is selected from the group consisting of G6 mutant T7, H9 mutant T7, H10 mutant T7. C4 mutant 17, consensus T7, Lac, Lac UV5, tac, trc, GAP, and xylA promoter.

### Kits

Provided is a transfection kit comprising an expression vector as described herein. Such a kit may comprise a carrying means being compartmentalized to receive in close confinement one or more container means such as, e.g., vials or test tubes. Each of such container means comprises components or a mixture of components needed to perform a transfection. Such kits may include, for example, one or more components selected from vectors, cells, reagents, lipid-aggregate forming compounds, transfection enhancers, or biologically active molecules.

### II. Methods, Vectors, Host Cells and Kits for the Production of Norbaeocystin

### Methods

Provided is a method for the production of norbaeocystin comprising contacting a prokaryotic host cell with one or more expression vectors, wherein each expression vector comprises a psilocybin production gene selected from the group consisting of psiD and psiK and combinations thereof; and culturing the host cell; wherein at least one psilocybin production gene is from Psilocybe cyanescens, Panaeolus cyanescens, Gymnopilus dilepis, or Gymnopilus junonius. In some embodiments, at least one expression vector further comprises a psilocybin production gene selected from the group consisting of psiD and psiK and combinations thereof from Psilocybe cubensis. In some embodiments, the prokaryotic host cell is further contacted with at least one expression vector comprising a psilocybin production gene selected from the group consisting of psiD and psiK and combinations thereof from Psilocybe cubensis.

In certain embodiments, none of the expression vectors comprises psiM.

In certain embodiments, the psiD gene encodes a polypeptide comprising the amino acid sequence of SEQ ID NO: 18, 26, 32, or 38, or a sequence having at least 60%, at least 70%, at least 80%, at least 90%, at least 95%, at least 96%, at least 97%, at least 98%, or at least 99% sequence identity thereto. In certain embodiments, the psiD comprises the amino acid sequence of Genbank accession number PPQ70875, KY984104, KY984101.1, PPQ80975, or a sequence having at least 60%, at least 70%, at least 80%, at least 90%, at least 95%, at least 96%, at least 97%, at least 98%, or at least 99% sequence identity thereto. In certain embodiments, the psiD is encoded by a nucleotide sequence comprising SEQ ID NO: 19, 27, 33, or 39, or a sequence having at least 60%, at least 70%, at least 80%, at least 90%, at least 95%, at least 96%, at least 97%, at least 98%, or at least 99% sequence identity thereto.

In certain embodiments, the psiK gene encodes a polypeptide comprising the amino acid sequence of SEQ ID NO: 20, 28, 34, or 40, or a sequence having at least 60%, at least 70%, at least 80%, at least 90%, at least 95%, at least 96%, at least 97%, at least 98%, or at least 99% sequence identity thereto. In certain embodiments, the psiK comprises the amino acid sequence of Genbank accession number PPQ70874, KY984102, KY984099.1, PPQ98758, or a sequence having at least 60%, at least 70%, at least 80%, at least 90%, at least 95%, at least 96%, at least 97%, at least 98%, or at least 99% sequence identity thereto. In certain embodiments, the psiK is encoded by a nucleotide sequence comprising SEQ ID NO: 21, 29, 35, or 41, or a sequence having at least 60%, at least 70%, at least 80%, at least 90%, at least 95%, at least 96%, at least 97%, at least 98%, or at least 99% sequence identity thereto.

In certain embodiments, the recombinant prokaryotic cell is selected from the group consisting of Escherichia coli, Corynebacterium glutamicum, Vibrio natriegens, Bacillus subtilis, Bacillus megaterium, Escherichia coli Nissle 1917, Clostridium acetobutlyicum, Streptomyces coelicolor, Lactococcus lactis, Pseudomonas putida, Streptomyces clavuligerus, and Streptomyces venezuelae.

In certain embodiments, the prokaryotic cell is contacted with an expression vector comprising a psilocybin production gene selected from the group consisting of a psiD gene, a psiK gene, and combinations thereof, all under control of a single promoter in operon configuration: wherein at least one psilocybin production gene is from Psilocybe cyanescens, Panaeolus cyanescens, Gymnopilus dilepis, or Gymnopilus junonius. In some embodiments, at least one psilocybin production gene is from Psilocybe cubensis.

In certain embodiments, the prokaryotic cell is contacted with an expression vector comprising a psiD gene and a psiK gene, wherein each gene is under control of a separate promoter in pseudooperon configuration; wherein at least one psilocybin production gene is from Psilocybe cyanescens, Panaeolus cyanescens, Gymnopilus dilepis, or Gymnopilus junonius. In some embodiments, at least one psilocybin production gene is from Psilocybe cubensis.

In certain embodiments, each gene is in monocistronic configuration, wherein each gene has a promoter and a terminator. Any configuration or arrangement of promoters and terminators is envisaged. In certain embodiments, none of the expression vectors comprises a psiM gene.

In some embodiments, the promoter is selected from the group consisting of G6 mutant T7, H9 mutant T7, H10 mutant T7, C4 mutant 17, consensus T7, Lac, Lac UV5, tac, trc, GAP, and xylA promoter.

In certain embodiments, the host cell is cultured with a supplement independently selected from the group consisting of 4-hydroxyindole, serine, methionine, 4-hydroxytryptophan, 4-hydroxytryptamine, and combinations thereof. In certain exemplary embodiments, the supplement is fed continuously to the host cell. In further embodiments, the host cell is grown in an actively growing culture. Continuous feeding is accomplished by using a series of syringe and/or peristaltic pumps whose outlet flow is directly connected to the bioreactor. The set point of these supplement addition pumps is adjusted in response to real-time measurement of cell biomass and specific metabolic levels using UV-vis absorption and HPLC analysis, respectively. The fed-batch fermentation process is focused on maximizing production of target metabolites through harnessing the ability of an actively growing and replicating cell culture to regenerate key co-factors and precursors which are critical to the biosynthesis of target metabolites. This process notably does not involve the centrifugal concentration and reconstitution of cell biomass to artificially higher cell density and/or into production media that was not used to build the initial biomass. The production process involves the inoculation of the reactor from an overnight preculture at low optical density, followed by exponential phase growth entering into a fed-batch phase of production, culminating in a high cell density culture.

The norbaeocystin is found extracellularly in the fermentation broth. In certain embodiments, the norbaeocystin is isolated. Norbaeocystin can be collected through drying the fermentation broth after centrifugation to remove the cell biomass. The resulting dry product can be extracted to further purify the norbaeocystin. Alternatively, the norbaeocystin can be extracted from the liquid cell culture broth using a solvent which is immiscible with water and partitions norbaeocystin into the organic phase. Furthermore, contaminants from the fermentation broth can be removed through extraction leaving the norbacocystin in the aqueous phase for collection after drying or crystallization procedures.

In certain embodiments, the methods described herein result in a titer of norbaeocystin of about 0.1 to about 50 g/L. In some embodiments, the methods described herein result in a titer of norbaeocystin of about 0.1 to about 12 g/L. In further embodiments, the methods described herein result in a titer of norbaeocystin of about 0.1 to about 6 g/L. In further embodiments, the methods described herein result in a titer of norbaeocystin of about 0.5 to about 3 g/L. In yet further embodiments, the methods described herein result in a titer of norbaeocystin of abou 1.5 g/L.

In certain embodiments, the methods described herein result in a molar yield of norbaeocystin of about 10% to about 100%. In some embodiments, the methods described herein result in a molar yield of norbaeocystin of about 20% to about 80%. In yet further embodiments, the methods described herein result in a molar yield of norbaeocystin of about 30% to about 70%. In certain embodiments, the methods described herein result in a molar yield of norbaeocystin of about 40% to about 60%. In further embodiments, the methods described herein result in a molar yield of norbaeocystin of about 50%.

### Recombinant Prokaryotic Cells for the Production of Norbaeocystin

Provided is a recombinant prokaryotic cell comprising one or more expression vectors, wherein each expression vector comprises a psilocybin production gene selected from the group consisting of psiD, psiK, and combinations thereof; wherein at least one psilocybin production gene is from Psilocybe cyanescens, Panaeolus cyanescens, Gymnopilus dilepis, or Gymnopilus junonius. In some embodiments, at least one psilocybin production gene is from Psilocybe cubensis. In certain embodiments, none of the expression vectors comprises psiM.

In certain embodiments, the recombinant prokaryotic cell is selected from the group consisting of Escherichia coli, Corynebacterium glutamicum, Vibrio natriegens, Bacillus subtilis, Bacillus megaterium, Escherichia coli Nissle 1917, Clostridium acetobutlyicum, Streptomyces coelicolor, Lactococcus lactis, Pseudomonas putida, Streptomyces clavuligerus, and Streptomyces venezuelae.

In certain embodiments, the psiD comprises the amino acid sequence of SEQ ID NO: 18, 26, 32, or 38, or a sequence having at least 60%, at least 70%, at least 80%, at least 90%, at least 95%, at least 96%, at least 97%, at least 98%, or at least 99% sequence identity thereto. In certain embodiments, the psiD comprises the amino acid sequence of Genbank accession number PPQ70875, KY984104, KY984101.1. PPQ80975, or a sequence having at least 60%, at least 70%, at least 80%, at least 90%, at least 95%, at least 96%, at least 97%, at least 98%, or at least 99% sequence identity thereto. In certain embodiments, the psiD is encoded by a nucleotide sequence comprising SEQ ID NO: 19, 27, 33, or 39, or a sequence having at least 60%, at least 70%, at least 80%, at least 90%, at least 95%, at least 96%, at least 97%, at least 98%, or at least 99% sequence identity thereto.

In certain embodiments, the psiK comprises the amino acid sequence of SEQ ID NO: 20, 28, 34, or 40, or a sequence having at least 60%, at least 70%, at least 80%, at least 90%, at least 95%, at least 96%, at least 97%, at least 98%, or at least 99% sequence identity thereto. In certain embodiments, the psiK comprises the amino acid sequence of Genbank accession number PPQ70874, KY984102, KY984099.1. PPQ98758, or a sequence having at least 60%, at least 70%, at least 80%, at least 90%, at least 95%, at least 96%, at least 97%, at least 98%, or at least 99% sequence identity thereto. In certain embodiments, the psiK is encoded by a nucleotide sequence comprising SEQ ID NO: 21, 29, 35, or 41, or a sequence having at least 60%, at least 70%, at least 80%, at least 90%, at least 95%, at least 96%, at least 97%, at least 98%, or at least 99% sequence identity thereto.

In certain embodiments, the prokaryotic cell is contacted with an expression vector comprising a psiD gene and a psiK gene all under control of a single promoter in operon configuration; wherein at least one psilocybin production gene is from Psilocybe cyanescens, Panaeolus cyanescens, Gymnopilus dilepis, or Gymnopilus junonius. In some embodiments, at least one psilocybin production gene is from Psilocybe cubensis. In certain embodiments, the prokaryotic cell is contacted with an expression vector comprising a psiD gene and a psiK gene, wherein each gene is under control of a separate promoter in pseudooperon configuration; wherein at least one psilocybin production gene is from Psilocybe cyanescens, Panaeolus cyanescens, Gymnopilus dilepis, or Gymnopilus junonius. In some embodiments, at least one psilocybin production gene is from Psilocybe cubensis. In certain embodiments, each gene is in monocistronic configuration, wherein each gene has a promoter and a terminator. Any configuration or arrangement of promoters and terminators is envisaged. In certain embodiments, none of the expression vectors comprises a psiM gene.

In some embodiments, the promoter is selected from the group consisting of G6 mutant T7, H9 mutant T7. H10 mutant T7. C4 mutant T7, consensus T7, Lac, Lac UV5, tac, trc, GAP, and xylA promoter.

### Expression Vectors

Provided is a vector for introducing at least one gene associated with psilocybin production; the gene may be selected from: psiD, psiK, and combinations thereof; wherein at least one psilocybin production gene is from Psilocybe cyanescens, Panaeolus cyanescens, Gymnopilus dilepis, or Gymnopilus junonius. In some embodiments, at least one psilocybin production gene is from Psilocybe cubensis.

In certain embodiments, the psiD gene encodes a polypeptide comprising the amino acid sequence of SEQ ID NO: 18, 26, 32, or 38, or a sequence having at least 60%, at least 70%, at least 80%, at least 90%, at least 95%, at least 96%, at least 97%, at least 98%, or at least 99% sequence identity thereto. In certain embodiments, the psiD comprises the amino acid sequence of Genbank accession number PPQ70875, KY984104, KY984101.1. PPQ80975, or a sequence having at least 60%, at least 70%, at least 80%, at least 90%, at least 95%, at least 96%, at least 97%, at least 98%, or at least 99% sequence identity thereto. In certain embodiments, the psiD is encoded by a nucleotide sequence comprising SEQ ID NO: 19, 27, 33, or 39, or a sequence having at least 60%, at least 70%, at least 80%, at least 90%, at least 95%, at least 96%, at least 97%, at least 98%, or at least 99% sequence identity thereto.

In certain embodiments, the psiK gene encodes a polypeptide comprising the amino acid sequence of SEQ ID NO: 20, 28, 34, or 40, or a sequence having at least 60%, at least 70%, at least 80%, at least 90%, at least 95%, at least 96%, at least 97%, at least 98%, or at least 99% sequence identity thereto. In certain embodiments, the psiK comprises the amino acid sequence of Genbank accession number PPQ70874, KY984102, KY984099.1. PPQ98758, or a sequence having at least 60%, at least 70%, at least 80%, at least 90%, at least 95%, at least 96%, at least 97%, at least 98%, or at least 99% sequence identity thereto. In certain embodiments, the psiK is encoded by a nucleotide sequence comprising SEQ ID NO: 21, 29, 35, or 41, or a sequence having at least 60%, at least 70%, at least 80%, at least 90%, at least 95%, at least 96%, at least 97%, at least 98%, or at least 99% sequence identity thereto.

In certain embodiments, the prokaryotic cell is contacted with an expression vector comprising a psiD gene and a psiK gene all under control of a single promoter in operon configuration: wherein at least one psilocybin production gene is from Psilocybe cyanescens, Panaeolus cyanescens, Gymnopilus dilepis, or Gymnopilus junonius. In some embodiments, at least one psilocybin production gene is from Psilocybe cubensis. In certain embodiments, the prokaryotic cell is contacted with an expression vector comprising a psiD gene and a psiK gene, wherein each gene is under control of a separate promoter in pseudooperon configuration; wherein at least one psilocybin production gene is from Psilocybe cyanescens, Panaeolus cyanescens, Gymnopilus dilepis, or Gymnopilus junonius. In some embodiments, at least one psilocybin production gene is from Psilocybe cubensis. In certain embodiments, each gene is in monocistronic configuration, wherein each gene has a promoter and a terminator. Any configuration or arrangement of promoters and terminators is envisaged. In certain embodiments, none of the expression vectors comprises a psiM gene.

In some embodiments, the promoter is selected from the group consisting of G6 mutant 17, H9 mutant 17, H10 mutant T7, C4 mutant T7, consensus T7, Lac, Lac UV5, tac, trc, GAP, and xylA promoter.

### Kits

Provided is a transfection kit comprising an expression vector as described herein. Such a kit may comprise a carrying means being compartmentalized to receive in close confinement one or more container means such as, e.g., vials or test tubes. Each of such container means comprises components or a mixture of components needed to perform a transfection. Such kits may include, for example, one or more components selected from vectors, cells, reagents, lipid-aggregate forming compounds, transfection enhancers, or biologically active molecules

### EXAMPLES

### Methods:

Strains. Plasmids. and Media

E. coli DH5a was used to propagate all plasmids, while BL21 Star™ (DE3) was the host for chemical production. Andrew's Magic Media (AMM) supplemented with 1 g/L methionine was used for all production experiments while Luria Broth (LB) was used for plasmid propagation during cloning.

### Gene Sourcing

Norbaeocystin methyltransferase sequences for Psilocybe cubensis (ASU62238), Psilocybe cyanescens (A0A409WXG9), Panaeolus cyanescens (A0A409WR68), and Gymnopilus dilepis (A0A409VX92), were sourced from UniprotKB via percent identity clusters. After obtaining both nucleotide and amino acid sequences, all of the latter were aligned using Clustal Omega Multiple Sequence Alignment. This resulted in the identification of conserved regions between mushroom species. These conserved regions were used to screen thousands of hypothetical proteins from a Gymnopilus junonius genomic sequence via GenBank (Accession: KAF8874878).

### Plasmid and Library Construction

The psiM gene sequences from each mushroom species was ordered as linear double stranded DNA from Genewiz. The template sequences were PCR amplified using primers 1-10, Table 5, digested with NdeI and XhoI, gel extracted, and ligated into the pETM6-SDM2x plasmid backbone also digested with NdeI and XhoI to create plasmids 1-5, Table 4. Seven plasmids (6-12, Table 4), each containing a different promoter sequence were pooled in equimolar quantities, digested with XbaI and ApaI, gel extracted, and ligated with the similarly digested pNor plasmid to create a pETM6-XX⁷-PsiDK plasmid library. The XX⁷ plasmid library was then digested with BcuI and ApaI and ligated with the respective psiM plasmid cut with XmaJI and ApaI. This resulted in five independent psilocybin pathway libraries with different psiM genes, each cloned in operon format. These ligated plasmid libraries were transformed into DH5α on ampicillin agar plates, scraped with a clean razor blade to pool all variants, and DNA was extracted from the resulting cell pellet in alignment with previously published methods¹¹. The purified plasmid library was validated by restriction digestion and transformed into BL21Star™(DE3). This library construction process was performed individually for the Gymnopilus dilepis (Gymdi), Gymnopilus junonius (Gymju), Panaeolus cyanescens (Pancy). Psilocybe cyanescens (Psicy), and Psilocybe cubensis (Psicu) psiM genes, creating five separate operon production libraries.

### Small Scale Fermentation Screening and Strain Validation

Library screening was performed in 2 mL cultures in 48-well plates at 37° C. AMM supplemented with methionine (1 g/L), 4-hydroxyindole (350 mg/L), and ampicillin (80 μg/mL) was used. Overnight cultures were grown from either an agar plate or freezer stock culture in AMM with appropriate antibiotics and supplements for 8 h in a shaking 37° C. incubator. Although some promoters under investigation were constitutive, induction occurred for all variants 4 h after inoculation with 1 mM isopropylβ-D-1-thiogalactopyranoside (IPTG). Cultures were then sampled 24 h post inoculation and subjected to HPLC-MS analysis for quantification of target metabolites.

### Bioreactor Scale-Up

Selected top-producing strains for both psilocybin and baeocystin were investigated for scale-up viability using a 1.5 L working volume in Eppendorf BioFlo120 bioreactors as described previously⁸. Fermentation conditions remained at 37° C. with AMM supplemented with serine (5 g/L), 4-hydroxyindole, ampicillin (80 μg/mL), and methionine supplementation appropriate for the desired product (0 g/L for baeocystin and 5 g/L for psilocybin). Overnight cultures were grown in a shaking 37° C. incubator for 12 h, or to an OD600 of at least 3.0, then added to the reactor at 2% v/v. Throughout the fermentation, glucose was fed using a 50% glucose feed solution in water, pH was maintained at 6.5 with 10M KOH, and the 4-hydroxyindole feed was varied constantly to control the buildup of the toxic intermediate, 4-hydroxytryptophan. Bioreactor samples were analyzed using HPLC for intermediate and final product titer, as well as glucose and fermentation by products (e.g., acetate) as described below.

### Analytical Methods

Metabolite analysis was performed on a Thermo Scientific Ultimate 3000 High-Performance Liquid Chromatography (HPLC) system equipped with Diode Array Detector (DAD), Refractive Index Detector (RID), and Thermo Scientific ISQ™ EC single quadrupole mass spectrometer (MS). Samples were prepared for HPLC and LC-MS analysis by centrifugation at 15,000×g for 5 min. A volume of 2 μL of the resulting supernatant was then injected for HPLC and LC-MS analysis. Authentic Standards were purchased for psilocybin (Cerilliant). Norbacocystin and baeocystin were quantified using standard produced, purified, and characterized in house.

Quantification of aromatic metabolites was performed using absorbance at 280 nm from the DAD and the metabolites were separated using an Agilent Zorbax Eclipse XDB-C18 analytical column (3.0 mm×250 mm, 5 μm) with mobile phases of water (A) and acetonitrile (B) both containing 0.1% formic acid at a total flow rate of 1 mL/min: 0 min, 5% B: 0.43 min, 5% B; 5.15 min, 19% B: 6.44 min, 100% B; 7.73 min, 100% B; 7.73 min, 5% B: 9.87 min, 5% B. This method resulted in the following observed retention times as verified by analytical standards (when commercially available) and MS analysis (as described below): 4-hydroxyindole (6.6 min), 4-hydroxytryptophan (3.4 min), 4-hydroxytryptamine (3.2 min), norbaeocystin (1.6 min), baeocystin (1.9 min) and psilocybin (2.2 min). A Bio-Rad Aminex HPX-87H column coupled with a RI detector was used for quantification of sugars and organic acids.

Liquid Chromatography Mass Spectrometry (LC-MS) data was collected where the full MS scan was used to provide an extracted ion chromatogram (EIC) of our compounds of interest. Analytes were measured in positive ion mode at the flow rate, solvent gradient, and column conditions described above. The instrument was equipped with a heated electrospray ionization (HESI) source and supplied ≥99% purity nitrogen from a Peak Scientific Genius XE 35 laboratory nitrogen generator. The source and detector conditions were as follows: sheath gas pressure of 80.0 psig, auxiliary gas pressure of 9.7 psig, sweep gas pressure of 0.5 psig, foreline vacuum pump pressure of 1.55 Torr, vaporizer temperature of 500° C., ion transfer tube temperature of 300° C., source voltage of 3049 V. and source current of 15.90 μA. Error bars represent +/−1 standard deviation from the mean of biological duplicates.

### Example 1: Methyltransferase Selection and Alignment Comparisons

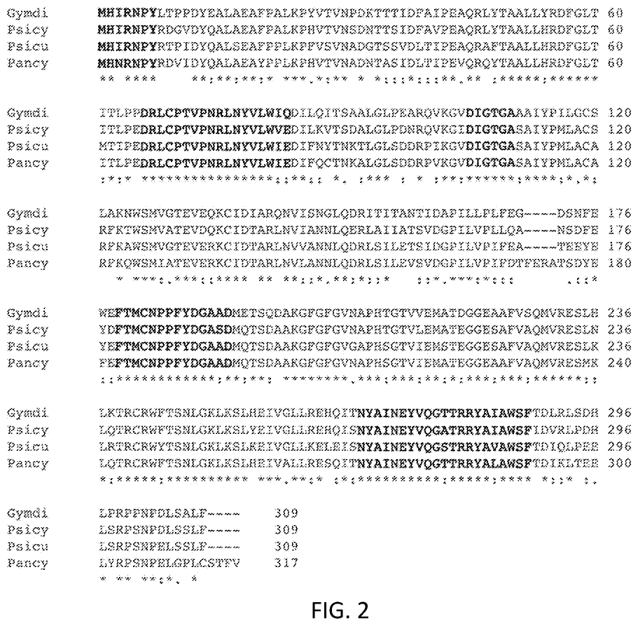

Amino acid sequence alignment¹¹ in FIG. 2 yielded a collection of conserved regions hypothesized to be integral to the enzyme's methylation activity. The percent identity matrix (Table 1) revealed that Psilocybe cyanescens, Psilocybe cubensis, and Panaeolus cyanescens varied very little, with all exact alignment scores over 80%.

TABLE 1
Amino Acid Identity Matrix for PsiM from Psilocybe cubensis
(Psicu), Psilocybe cyanescens (Psicy), Panaeolus cyanescens
(Pancy), Gymnopilus dilepis (Gymdi), and Gymnopilus junonius
(Gymju). Created by Clustal12.1

| | Gymju | Gymdi | Psicy | Psicu | Pancy |
|---|---|---|---|---|---|
| Gymnopilus junonius | 100.0 | 49.2 | 50.2 | 49.2 | 48.8 |
| Gymnopilus dilepis | 49.2 | 100.0 | 78.3 | 71.2 | 73.1 |
| Psilocybe cyanescens | 50.2 | 78.3 | 100.0 | 77.7 | 79.3 |
| Psilocybe cubensis | 49.2 | 71.2 | 77.7 | 100.0 | 83.8 |
| Panaeolus cyanescens | 48.8 | 72.9 | 79.29 | 83.8 | 100.0 |

In contrast, large amino acid sequence variation was found in the Gymnopilus genus; the junonius and dilepis species shared only 48% identity. Although not wholly conserved, a majority of the amino acids within the previously identified conserved regions were maintained¹². When Gymnopilus junonius is aligned pairwise in comparison to Psilocybe cubensis alone, the similarity is 67% with an additional 60 amino acids exhibiting similar biochemical properties¹³. While the junonius species is noticeably less related to the other 4 methyltransferases, the percent identity among all 4 are almost identical at around 47% (Table 1). Due to the sourcing of the junonius methyltransferase, truncating the large 3′ region of the protein sequence to align with those previously identified may be considered.

### Example 2: Norbaeocystin Uptake

A pETM6-SDM2x plasmid backbone was ligated with the psiM genes of interest, verified through restriction digest, and transformed into the production strain BL21Star™(DE3). These strains then underwent activity screenings in monoculture with a norbaeocystin supplement and via co-culture with a previously optimized norbaeocystin production strain, pNor, and a 4-hydroxyindole supplement. In the co-culture screening, the ratio of pNor to psiM inoculum was varied including a 1:1, 1:4, and 1:9 to account for the variance in functional activity of the two modules. The strain ratios were skewed towards an excess of the psiM-expressing strain to account for the fact that pNor had previously been optimized and likely would outperform the newly constructed psiM variants. Both experimental setups resulted in the expected amount of norbaeocystin availability but exhibited no psilocybin production. Without wishing to be bound by theory, this suggests that the cell may exhibit an inability to reuptake norbaeocystin into the cytoplasm in order to facilitate methylation. These surprising preliminary results necessitated a plasmid construct that contains all three genes in the exogenous pathway to circumvent any intermediate uptake issues. Furthermore, transcriptional libraries of these new pathway constructs would need to be screened to fully evaluate their potential and to enable a fair comparison between variants.

### Example 3: Monoculture Library Screening Yielded a Range of Valuable Production Strains

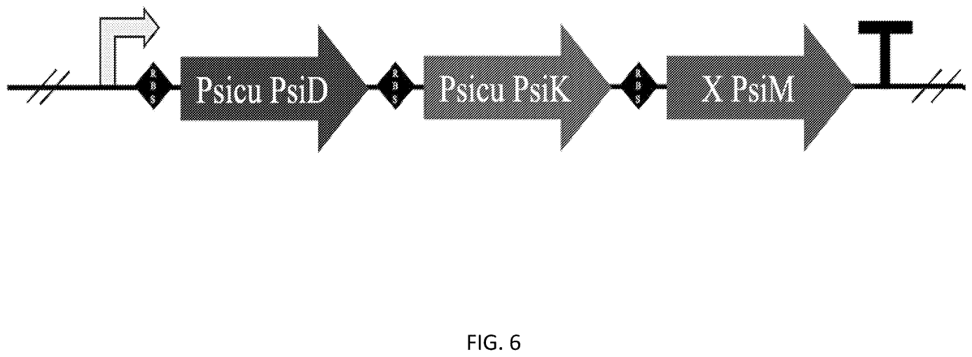

Utilizing psiD and psiK from Psilocybe cubensis and psiM from Psilocybe cubensis, Psilocybe cyanescens, Panaeolus cyanescens. Gymnopilus dilepis, and Gymnopilus junonius, five independent transcriptionally varied libraries were cloned in operon configuration (FIG. 6), each with seven possible promoters: H9, H10, C4, G6, pXylA, pGAP, and the T7 consensus (Table 2). This configuration allowed for a possible library size of seven, for each of the five pathway configurations. Initial screening of 3× library size in high throughput 48-well assays yielded select strains of interest. All mutants were then selected for further experimentation based on their percentage of baeocystin production or overall psilocybin titer.

TABLE 2
Sequence of T7 consensus and mutant T7 promoters. Regions involved with T7-
RNA polymerase binding specificity and strength are marked for reference.
Bolded region specifies mutation region.

| Construct Name | Mutant T7 Promoter Sequence | SEQ ID NO. | Strength |
|---|---|---|---|
| Consensus | TAATACGACTCACTATAGGGGAA | 1 | High |
| C4 | TAATACGACTCACTATCAAGGAA | 2 | High |
| G6 | TAATACGACTCACTATTTCGGAA | 3 | Low |
| H9 | TAATACGACTCACTAATACTGAA | 4 | Med/Low |
| H10 | TAATACGACTCACTACGGAAGAA | 5 | Medium |

Pathways containing Gymnopilus dilepis PsiM (Gymdi) presented many strains of interest, with three randomly selected colonies producing an average of 507.4±8.8 mg/L of psilocybin. These strains outperformed the previously established psilocybin production strain, pSilo16, under identical conditions⁷ by 270%. (pSilo16 is the same as pPsilo16 in WO 2021/086513, which is hereby incorporated by reference in its entirety). Although able to produce large amounts of psilocybin, none of the examined Gymdi configurations resulted in specific compositional enhancements to any of the other methylated products. The highest baeocystin titer from this strain was only 69.9 mg/L. While a low concentration compared to psilocybin production from top Gymdi strains, taken alone, it was a high titer for a preliminary screen to create a strain capable of producing baeocystin in high quantities. Three Gymdi strains were selected for further study: one for high baeocystin titer and two for high psilocybin titer.

Pathways containing Psilocybe cubensis PsiM (Psicu) acted as a baseline for this experiment as these libraries have been similarly constructed in a previous study⁸. We selected 1 high producing and 1 low producing strain to verify the previously discovered promoter configurations, confirming the validity and success of the screening and selection approach.

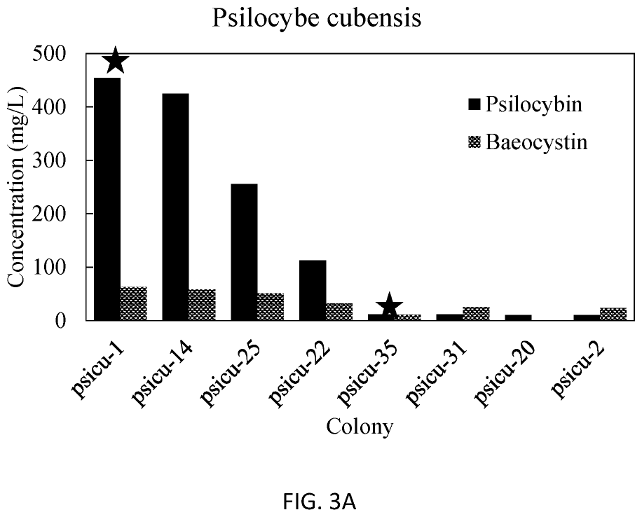

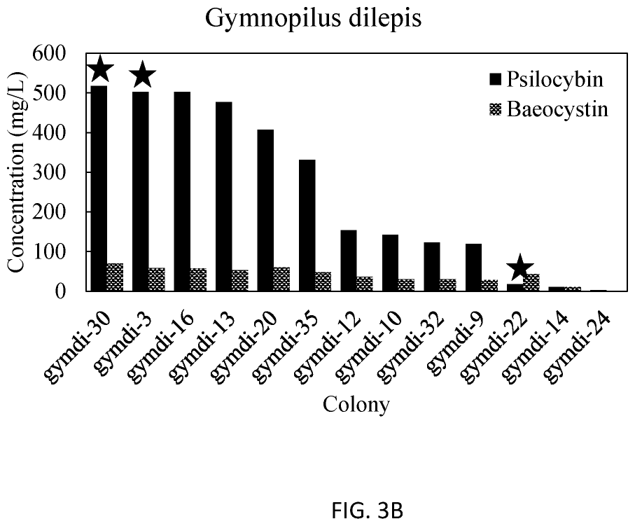

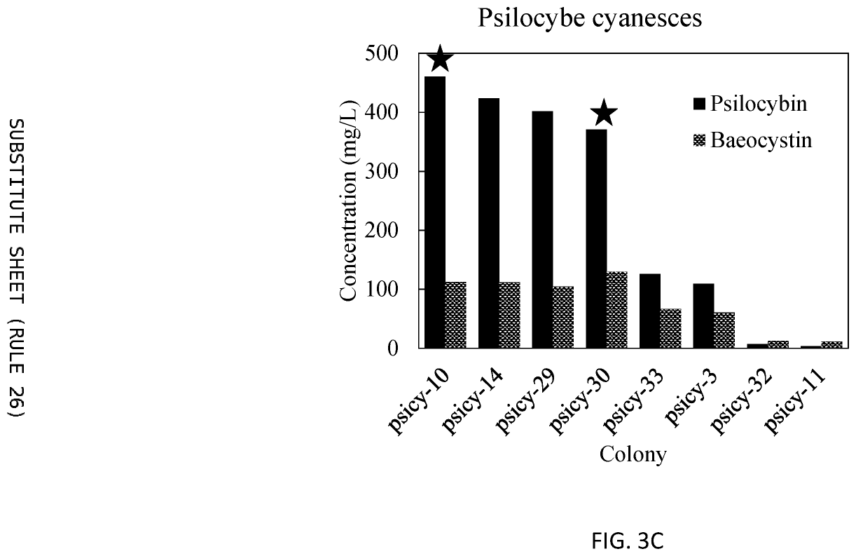

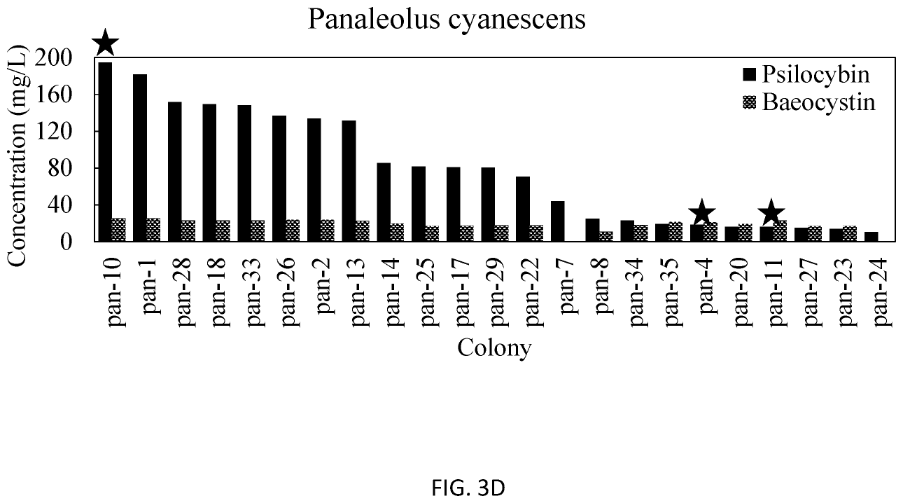

Pathways containing Psilocybe cyanescens PsiM (Psicy) displayed an overall limited number of productive mutants, however, one mutant was selected as psilocybin overproducer, while another demonstrated the highest baeocystin titer observed in our preliminary screen and was also selected for further screening. FIG. 3C. Top producing mutants containing Panaeolus cyanescens PsiM (Pancy) showed muted production compared to those from other PsiM libraries. The Pancy library contained a few notable mutants with higher baeocystin production than psilocybin, however, the absolute titers in this case were low in comparison to lead baeocystin-production mutants from other libraries (FIG. 3D). We selected 3 mutants from this library: the highest psilocybin producer and 2 mutants with enhanced baeocystin fraction, despite low overall production.

### Example 4: Rescreening of Lead Mutants Resulted in Confirmation of Metabolite Production

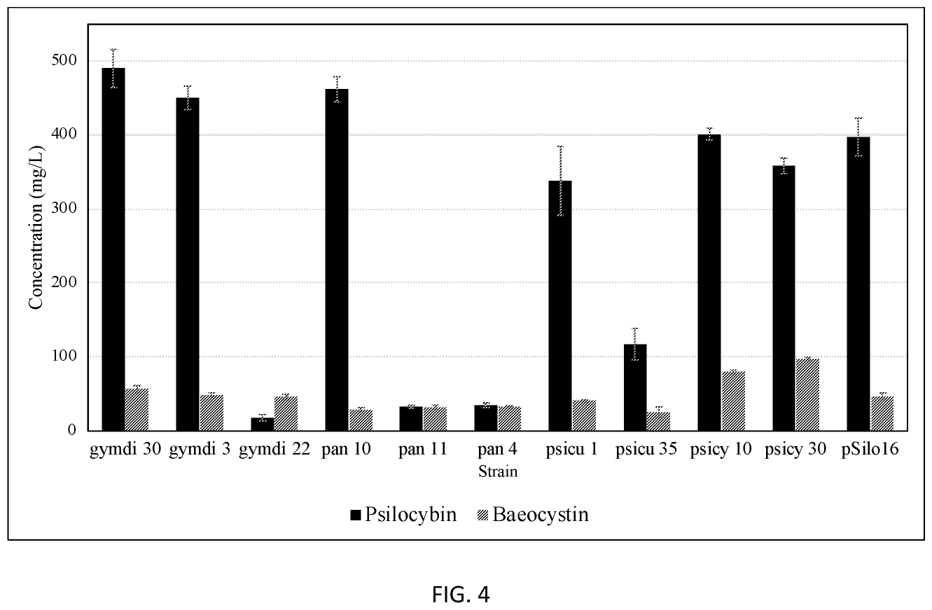

Selected mutants were run in duplicate under identical fermentation conditions. The 48-well plates were incubated for two days before data collection with HPLC. Data was analyzed for bacocystin, psilocybin, psilocin, and aeruginascin. FIG. 4 demonstrates the concentration of all metabolites found, not including aeruginascin or psilocin as no significant accumulation was observed, as consistent with previous studies in E. coli⁸. Plasmid DNA containing the production pathway from each isolated mutant was purified and sent for sequencing to confirm the promoter controlling exogenous gene expression (Table 3). Both high and low Psicu producers were selected for sequencing to verify the medium throughput library cloning, screening, and selection processes were capable of reproducing previously identified high and low psilocybin producers.⁸

TABLE 3
Promoter Validation

| | | | Relative |
|---|---|---|---|
| Strain Name | Promoter | Type | Strength |
| Pancy 11-1 | C4 | Inducible | High |
| Psicy 10-4 | pXylA | Constitutive | Low |
| Gymdi 22-3 | T7 | Inducible | High |
| Gymdi 23-4 | C4 | Inducible | High |
| Psicu 6-3 | G6 | Inducible | Low |
| Psicu 12-1 | C4 | Inducible | High |

In each of the transcriptional libraries screened, a wide variety of metabolite concentrations and compositions were observed. In multiple instances, the metabolite concentrations varied by nearly two orders of magnitude, while the bacocystin composition ranged from 10% to 90% of the total methylated tryptamines. Data suggests that norbaeocystin methyltransferases showed less of an affinity towards the first (baeocystin), or third methylation (aeruginascin). Instead the strains accumulating the highest concentrations of methylated tryptamines trended towards an accumulation of psilocybin. Strains exhibiting high baeocystin composition were most generally associated with lower overall tryptamine production, further complicating the search for a baeocystin over producing strain.

Upon promoter sequence analysis, we discovered several top psilocybin producing mutants (Psicy30, Gymdi30, and Pancy10) contained the low strength constitutive promoter, pXylA (Table 3). This was particularly interesting as all previous psilocybin producing E. coli strains contained IPTG-inducible T7 mutant promoters. Upon performing an economic analysis of psilocybin production cost via microbial fermentation. IPTG, was identified as the single most expensive required chemical component. Furthermore, the added process complexity of induction timing motivated the development and scaleup of constitutive expression psilocybin production strains as they represent a clear economic advantage over current technology.

The high sensitivity of these pathway variants to transcriptional balancing illustrates the need to evaluate new gene constructs under a variety of transcriptional environments to fully understand their potential. Furthermore, while varied promoter strengths change the transcriptional frequency of psiM production, they do not alter the sequence, structure, or mechanism of action of the PsiM enzyme. Further work must be completed to understand the rationale as to how the transcriptional strength of expression can contribute to the variation observed in product composition from a single enzyme (e.g., psilocybin vs. baeocystin vs. norbaeocystin). Consideration of holistic genetic and fermentation optimization approaches for this pathway may give us insight into the mechanistic rules governing pathway function.

### Example 5: Enhanced Psilocybin Production Via Bioreactor Scale Up

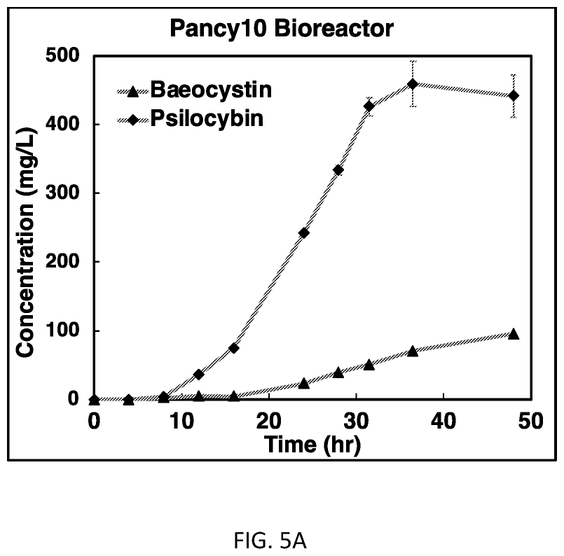

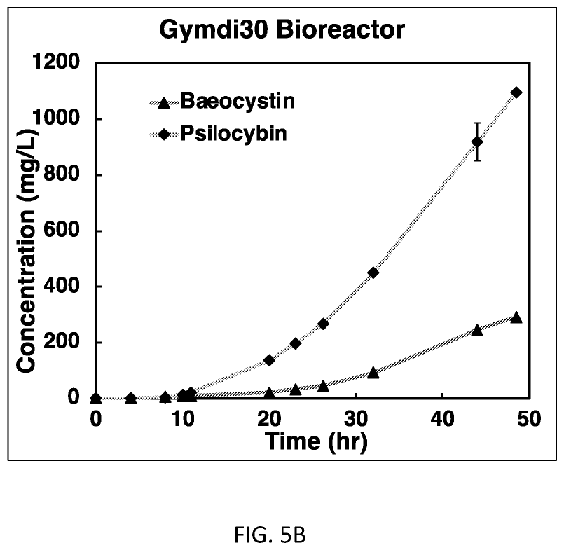

Two of the top psilocybin production strains, both with constitutive pathway expression, Gymdi30 and Pancy10, were investigated in 1.5 L working volume bioreactors under fed-batch conditions. Mutant validation of Pancy10 resulted in a psilocybin titer of 462.1±16.6 mg/L under small-scale batch fermentation. Upon scaleup, production of psilocybin did not increase as dramatically as was expected based on previous scale up experiments with psilocybin producing E. coli (FIG. 5). Gymdi30, however, averaged 490±25.7 mg/L of psilocybin before scale up, and yielded more than a 2.4-fold increase in production, with a final titer of 1.19 g/L under fed-batch conditions (FIG. 5). Additional studies are underway to further optimize and characterize bioreactor scale production for this elite production mutant. This work has created a psilocybin production strain comparable to previous top psilocybin production strains with the additional cost and process benefit of constitutive pathway expression.

### Example 6: Further Scale Un Studies

Scale up studies are performed with lead strains under a variety of media supplement conditions culminating with evaluation of top strains in an Eppendorf BioFlo120 bioreactor at 1.5 L working volume. Performance under pH and dissolved oxygen control with a continual feed of glucose and 4-hydroxyindole substrate is studied. Development of pseudooperon and monocistronic library configurations utilizing the newly sourced psiM, psiD and psiK enzyme variants is also conducted. Sequences for psiD, psiK, and psiM genes from various mushroom species are provided herewith.

### BIBLIOGRAPHY

1. “NIMH” Mental Illness.” National Institute of Mental Health, U.S. Department of Health and Human Services, www.nimh.nih.gov/health/statistics/mental-illness #:˜:text=Mental%20illnesses%20arc%20common%20in,mild%20to%20moderate%20to%20severe.
2. NHS Choices, NHS, www.nhs.uk/mental-health/talking-therapies-medicine-treatments/medicines-and-psychiatry/antidepressants/side-effects/.
3. Stephen Ross, Anthony Bossis. “Rapid and Sustained Symptom Reduction Following Psilocybin Treatment for Anxiety and Depression in Patients with Life-Threatening Cancer: A Randomized Controlled Trial—Stephen Ross, Anthony Bossis, Jeffrey Guss, Gabrielle Agin-Liebes, Tara Malone, Barry Cohen, Sarah E Mennenga, Alexander Belser, Krystallia Kalliontzi, James Babb, Zhe Su, Patricia Corby, Brian L Schmidt, 2016.” SAGE Journals, journals.sagepub.com/doi/full/10.1177/0269881116675512?url_ver=Z39.88-2003&rfr_id=ori%3Arid%3Acrossref.org&rfr_dat=cr_pub%3Dpubmed.
4. Jeffrey A. Gluff Jeffrey A. Gluff. “Post-Traumatic Stress Disorder (PTSD): A Webliography.” Taylor & Francis, www.tandfonline.com/doi/full/10.1080/15398285.2017.1377539.
5. Bauer, Barbara E. “The Entourage Effect in Magic Mushrooms.” Psychedelic Science Review, 17 Nov. 2020, psychedelicreview.com/the-entourage-effect-in-magic-mushrooms/.
6. Books on Drug Abuse, druglist.info/books-on-drug-abuse/.
7. “Analysis of Aeruginascin in Fruit Bodies of the Mushroom Inocybe aeruginascens.” Taylor & Francis, www.tandfonline.com/doi/abs/10.3109/13880208909053954.
8. Adams, Alexandra M., et al. “In Vivo Production of Psilocybin in E. coli.” Metabolic Engineering. Academic Press, 21 Sep. 2019, www.sciencedirect.com/science/article/pii/S109671761930309X?via%3Dihub#bib34.
9. Fricke, Janis, et al. “Enzymatic Synthesis of Psilocybin.” Wiley Online Library. John Wiley & Sons, Ltd, 25 Aug. 2017, onlinelibrary.wiley.com/doi/10.1002/anie.201705489.
10. Reynolds, Hannah T, et al. “Horizontal Gene Cluster Transfer Increased Hallucinogenic Mushroom Diversity.” Evolution Letters. John Wiley and Sons Inc., 27 Feb. 2018, www.ncbi.nlm.nih.gov/pmc/articles/PMC6121855/.
11. Madeira F, Park Y M, Lee J. et al. The EMBL-EBI search and sequence analysis tools APIs in 2019. Nucleic Acids Research. 2019 July; 47(W1):W636-W641. DOI: 10.1093/nar/gkz268. PMID: 30976793; PMCID: PMC6602479.
12. Pearson, William R. “[5] Rapid and Sensitive Sequence Comparison with FASTP and Fasta.” Methods in Enzymology, 1990, pp. 63-98., doi.org/10.1016/0076-6879(90)83007-v.
13. “Gymju to Psicu Piecewise Alignment.” EBI, www.ebi.ac.uk/Tools/services/rest/emboss_needle/result/emboss_needle-120211014-234542-0855-94997477-p2m/aln.
14. Jones, J. Andrew, et al. “EPathOptimize: A Combinatorial Approach for Transcriptional Balancing of Metabolic Pathways.” Scientific Reports, vol. 5, no. 1, 2015, doi.org/10.1038/srep 1301.
15. Xu, P., Vansiri, A., Bhan, N., & Koffas, M. A. (2012). cPathBrick: a synthetic biology platform for engineering metabolic pathways in E. coli. ACS synthetic biology. 1(7), 256-266.
16. Jones. J. A. Vemacchio, V. R., Collins, S. M. Shirke, A. N., Xiu, Y., Englaender, J. A., . . . & Koffas, M. A. (2017). Complete biosynthesis of anthocyanins using E. coli polycultures. MBio, 8(3), e00621-17.

TABLE 4
Strain List

| # | Plasmid | Description | Species Origin | References |
|---|---|---|---|---|
| 1 | pETM6-SDM2x- | psiD and psiK | Psilocybe | This Study |
| | pNor | source | cubensis | |
| 2 | pETM6-SDM2x- | methyltransferase | Gymnopilus | This Study |
| | GymdiPsiM | gene | dilepis | |
| 3 | pETM6-SDM2x- | methyltransferase | Gymnopilus | This Study |
| | GymjuPsiM | gene | junonius | |
| 4 | pETM6-SDM2x- | methyltransferase | Panaeolus | This Study |
| | PancyPsiM | gene | cyanescens | |
| 5 | pETM6-SDM2x- | methyltransferase | Psilocybe | This Study |
| | PsicuPsiM | gene | cubensis | |
| 6 | pETM6-SDM2x- | methyltransferase | Psilocybe | This Study |
| | PsicyPsiM | gene | cyanescens | |
| 7 | pETM6-H9- | Mutant T7 | — | [14] |
| | mCherry | promoter source | | |
| 8 | pETM6-H10- | Mutant T7 | — | [14] |
| | mCherry | promoter source | | |
| 9 | pETM6-C4- | Mutant T7 | — | [14] |
| | mCherry | promoter source | | |
| 10 | pETM6-G6- | Mutant T7 | — | [14] |
| | mCherry | promoter source | | |
| 11 | pETM6-T7- | T7 promoter | — | [15] |
| | mCherry | source | | |
| 12 | pETM6-pXylA- | Weak constitutive | — | [16] |
| | mCherry | promoter source | | |
| 13 | pETM6-pGAP- | Strong | — | [16] |
| | mCherry | constitutive | | |
| | | promoter source | | |
| 14 | pETM6-XX⁷- | Promoter library | Psilocybe | This Study |
| | PsicuDKPsicuM | for validation of | cubensis | |
| | | Psilocybe | | |
| | | cubensis psiM | | |
| 15 | pETM6-XX⁷- | Promoter library | Psilocybe | This Study |
| | PsicuDKPsicyM | for validation of | cubensis | |
| | | Psilocybe | Psilocybe | |
| | | cyanescens psiM | cyanescens | |
| 16 | pETM6-XX⁷- | Promoter library | Psilocybe | This Study |
| | PsicuDKPancyM | for validation of | cubensis | |
| | | Panaeolus | Panaeolus | |
| | | cyanescens psiM | cyanescens | |
| 17 | pETM6-XX⁷- | Promoter library | Psilocybe | This Study |
| | PsicuDKGymdiM | for validation of | cubensis | |
| | | Gymnopilus | Gymnopilus | |
| | | dilepis psiM | dilepis | |
| 18 | pETM6-XX⁷- | Promoter library | Psilocybe | This Study |
| | PsicuDKGymjuM | for validation of | cubensis | |
| | | Gymnopilus | Gymnopilus | |
| | | junonius psiM | junonius | |

TABLE 5
Primers for Methyltransferases

| | | | SEQ ID |
|---|---|---|---|
| | Primers | Sequence | NO. |
| 1 | Gymju_FWD | CGGCTCCATATGCACTCTCG | 8 |
| 2 | Gymju_REV | GAGCCGCTCGAGTTAACTGG | 9 |
| 3 | Gymdi_FWD | CGGCTCCATATGCACATCAGG | 10 |
| 4 | Gymdi_REV | GAGCCGCTCGAGCTAGAACAAAG | 11 |
| 5 | Pancy_FWD | CGGCTCCATATGCACAACAGAAACC | 12 |
| 6 | Pancy_REV | GAGCCGCTCGAGTCAGACAAAG | 13 |
| 7 | Psicy_FWD | CGGCTCCATATGCATATCAGGAACC | 14 |
| 8 | Psicy_REV | GAGCCGCTCGAGCTAGAAAAGAG | 15 |
| 9 | Psicu_FWD | GCCGCCCATATGCATATCAGAAATCCTTACCGTACAC | 16 |
| 10 | Psicu_REV | GGCGCGACTAGTCTAGAAAAGAGAGCTGAGCTCGGG | 17 |

TABLE 6
Sequences

| SEQ | | |
|---|---|---|
| ID | | |
| NO: | Description | Sequence |
| 1 | T7 Consensus | TAATACGACTCACTATAGGGGAA |
| | promoter | |
| 2 | C4 promoter | TAATACGACTCACTATCAAGGAA |
| 3 | G6 promoter | TAATACGACTCACTATTTCGGAA |
| 4 | H9 promoter | TAATACGACTCACTAATACTGAA |
| 5 | H10 promoter | TAATACGACTCACTACGGAAGAA |
| 6 | GAP | GCGTAATGCTTAGGCACAGGATTGATTTGTCGCAATGATTGA |
| | promoter | CACGATTCCGCTTGACGCTGCGTAAGGTTTTTGTAATTTTAC |
| | | AGGCAACCTTTTATTCA |
| 7 | XyIA | TTGAAATAAACATTTATTGTATATGATGAGATAAAGTTAGTT |
| | promoter | TATTGGATAAACAAACTAACTCAATTAAGATAGTTGATGGAT |
| | | AAACTT |
| 8 | Gymju FWD | CGGCTCCATATGCACTCTCG |
| 9 | Gymju_REV | GAGCCGCTCGAGTTAACTGG |
| 10 | Gymdi FWD | CGGCTCCATATGCACATCAGG |
| 11 | Gymdi_REV | GAGCCGCTCGAGCTAGAACAAAG |
| 12 | Pancy_FWD | CGGCTCCATATGCACAACAGAAACC |
| 13 | Pancy REV | GAGCCGCTCGAGTCAGACAAAG |
| 14 | Psicy_FWD | CGGCTCCATATGCATATCAGGAACC |
| 15 | Psicy_REV | GAGCCGCTCGAGCTAGAAAAGAG |
| 16 | Psicu FWD | GCCGCCCATATGCATATCAGAAATCCTTACCGTACAC |
| 17 | Psicu REV | GGCGCGACTAGTCTAGAAAAGAGAGCTGAGCTCGGG |
| 18 | Gymnopilus | MAKTLRPTAQAFRELGWLPASDGVYNKFMKDLTNRASNENHL |
| | dilepis | CHVALLQPIQDFKTFIENDPVVYQEFVCMFEGIEESPRNYHELC |
| | PsiD | NMFNEIFRRAPYYGDLGPPVYMAMAKIMNTRAGFSAFTRESLN |
| | Genbank | FHFKRLFDTWGLFLSSPASRDVLVADKFDSKHYGWFSEPAKAA |
| | Accession No. | MMAQYDGRTFEQVFICDETAPYHGFKSYDDFFNRKFRAMDID |
| | PPQ70875 | RPVVGGIANTTLIGSPCEALSYNVSDDVHSLETLYFKGEGYSLR |
| | | HLLHDDPSTEQFEHGSIIQGFLNITGYHRWHAPVSGTIMKIVDVP |
| | | GTYFAQAPSTIGDPFPVNDYDPQAPYLRSLAYFSNIAARQIIFIQ |
| 19 | Amino Acid | ADNEDIGLIYLILIGMTEVSTCEALVCPGQHVERGDDLGMFHFG |
| | Sequence | GSSFALGLRKNSKAAILEELKTQGTVIKVNDVIAAVQA |
| | Gymnopilus | ATGGCCAAAACGCTACGACCCACTGCCCAGGCCTTTCGAGA |
| | dilepis | ACTCGGTTGGCTGCCTGCCAGCGACGGAGTTTACAACAAGTT |
| | PsiD | CATGAAGGACTTGACGAATCGGGCCAGCAACGAAAATCACT |
| | Nucleotide | TATGCCATGTTGCCCTTCTGCAGCCCATCCAAGATTTCAAAA |
| | Sequence | CATTCATTGAGAACGATCCTGTTGTGTACCAGGAATTTGTTT |
| | | GCATGTTTGAGGGAATCGAGGAGTCTCCTAGAAATTATCATG |
| | | AGCTATGTAACATGTTCAACGAAATCTTCCGAAGGGCCCCAT |
| | | ATTACGGGGATCTAGGGCCTCCAGTGTACATGGCCATGGCTA |
| | | AAATTATGAATACGCGAGCTGGCTTCTCCGCATTCACAAGAG |
| | | AGAGCTTGAACTTCCACTTCAAAAGACTCTTCGATACTTGGG |
| | | GTTTATTCCTTTCCTCGCCAGCCTCACGCGACGTGCTTGTTGC |
| | | AGACAAGTTCGACAGCAAGCATTATGGCTGGTTTAGCGAAC |
| | | CTGCCAAGGCGGCTATGATGGCTCAATACGACGGACGTACA |
| | | TTTGAACAAGTCTTTATCTGCGACGAGACCGCTCCTTACCAC |
| | | GGCTTCAAATCTTACGACGACTTTTTCAACCGGAAATTCAGA |
| | | GCCATGGACATCGATCGCCCAGTCGTCGGTGGGATCGCCAA |
| | | CACTACCCTCATTGGGTCTCCTTGCGAAGCGTTGTCGTACAA |
| | | CGTCTCGGATGACGTCCATTCTTTGGAAACTCTGTACTTCAA |
| | | AGGCGAGGGTTATTCTCTCAGACACCTGCTCCACGACGATCC |
| | | TTCTACGGAACAGTTCGAGCATGGAAGTATTATTCAAGGATT |
| | | CCTCAACATCACTGGCTATCACCGATGGCACGCACCCGTGAG |
| | | TGGAACAATCATGAAGATCGTCGACGTCCCGGGCACCTACTT |
| | | CGCTCAGGCGCCCAGCACAATTGGAGATCCATTCCCAGTCA |
| | | ATGACTACGACCCGCAGGCTCCTTACCTCAGGTCTCTCGCAT |
| | | ACTTCTCCAACATTGCCGCCAGGCAGATTATCTTCATCCAAG |
| | | CCGACAACGAGGACATCGGCTTGATATATCTAATTCTAATCG |
| | | GTATGACGGAGGTCTCGACTTGCGAGGCCCTTGTGTGCCCTG |
| | | GTCAGCATGTCGAACGGGGCGACGATCTGGGAATGTTCCAT |
| | | TTCGGTGGTTCATCCTTCGCTCTTGGCCTTCGCAAGAACTCA |
| | | AAAGCCGCGATTCTCGAAGAACTCAAGACGCAGGGAACTGT |
| | | CATCAAAGTCAACGACGTGATAGCGGCTGTTCAAGCGTAA |
| 20 | Gymnopilus | MTFDLKTEEGLLVYLTQHLSLDVDLDGLKRLSGGFVNITWRIR. |
| | dilepis | LNAPFKGYTNIILKHAQPHLSSDENFKIGVERSAYEYRALKIVSE |
| | PsiK | SPILSGDDNLVFVPQSLHYDVVHNALIVQDVGSLKTLMDYVTA |
| | Genbank | RPSLSSEMAKLVGGQIGAFIARLHNIGRENKDHPEFNFFSGNIVG |
| | Accession No. | RTTAVQLYETIVPNATKYDIDDPIIPVVVQELIEEVKGSDETLIM |
| | PPQ70874 | ADLWGGNILLEFGKDSSDLGKIWVVDWELCKYGPPSLDMGYF |
| | Amino Acid | LGDCFLLAQFQDEKVATAMRRAYLENYAKIAKVPMDYDRSTT |
| | Sequence | GIGAHLVMWTDFMNWGSDEERKTSVEKGVRAFHDAKRDNKE |
| | | GEIPSILLRESSRT |
| 21 | Gymnopilus | ATGACTTTCGATCTCAAGACTGAAGAAGGCCTCTTAGTCTAT |
| | dilepis | CTTACTCAGCACCTATCGTTGGACGTCGACCTCGATGGGCTG |
| | PsiK | AAGCGTCTCAGCGGCGGCTTCGTCAACATCACCTGGCGGATT |
| | Nucleotide | AGACTCAACGCTCCTTTCAAAGGTTACACGAACATCATCTTG |
| | Sequence | AAGCACGCTCAGCCGCACTTATCGTCAGACGAGAATTTTAA |
| | | GATTGGGGTAGAGCGGTCGGCATACGAATATCGAGCACTGA |
| | | AAATCGTGTCTGAGAGTCCTATACTTAGCGGCGATGATAATC |
| | | TTGTCTTCGTACCTCAAAGTCTTCATTACGACGTCGTTCATAA |
| | | TGCCTTGATCGTGCAAGACGTGGGGTCGCTGAAGACCCTCAT |
| | | GGATTATGTCACGGCCAGACCGTCACTTTCATCGGAGATGGC |
| | | CAAGCTTGTCGGCGGTCAGATTGGTGCCTTCATCGCTCGACT |
| | | GCATAATATCGGACGCGAGAATAAGGACCATCCGGAATTCA |
| | | ATTTTTTCTCGGGAAACATCGTCGGAAGAACAACGGCTGTTC |
| | | AGCTATATGAAACCATCGTTCCCAACGCCACCAAGTACGAT |
| | | ATCGACGACCCGATTATTCCTGTAGTGGTTCAGGAGTTGATC |
| | | GAGGAAGTCAAAGGCAGCGACGAGACGCTTATAATGGCGGA |
| | | TCTGTGGGGTGGCAATATCCTTCTCGAGTTTGGGAAGGACTC |
| | | CTCGGATTTGGGAAAGATATGGGTCGTAGACTGGGAGTTAT |
| | | GCAAATACGGACCCCCTTCTTTGGACATGGGTTACTTCTTAG |
| | | GCGATTGTTTCCTTCTCGCTCAGTTTCAAGACGAAAAGGTCG |
| | | CGACGGCCATGAGAAGGGCCTACTTGGAGAATTACGCGAAG |
| | | ATTGCCAAGGTCCCAATGGACTATGATAGGAGCACGACAGG |
| | | CATTGGGGCGCATCTCGTCATGTGGACTGACTTCATGAATTG |
| | | GGGGAGCGATGAGGAAAGGAAGACGTCTGTGGAGAAGGGT |
| | | GTCAGGGCTTTCCATGATGCAAAGAGGGACAACAAGGAAGG |
| | | GGAAATTCCATCTATACTTTTGCGAGAATCGTCAAGAACGTA |
| | | G |
| 22 | Gymnopilus | MHIRNPYLTPPDYEALAEAFPALKPYVTVNPDKTTTIDFAIPEA |
| | dilepis | QRLYTAALLYRDFGLTITLPPDRLCPTVPNRLNYVLWIQDILQIT |
| | PsiM | SAALGLPEARQVKGVDIGTGAAAIYPILGCSLAKNWSMVGTEV |
| | Genbank | EQKCIDIARQNVISNGLQDRITITANTIDAPILLPLFEGDSNFEWE |
| | Accession No. | FTMCNPPFYDGAADMETSQDAKGFGFGVNAPHTGTVVEMATD |
| | PPQ70884 | GGEAAFVSQMVRESLHLKTRCRWFTSNLGKLKSLHEIVGLLRE |
| | Amino Acid | HQITNYAINEYVQGTTRRYAIAWSFTDLRLSDHLPRPPNPDLSA |
| | Sequence | LF |
| 23 | Gymnopilus | ATGCACATCAGGAATCCGTACCTCACTCCCCCAGACTACGAA |
| | dilepis | GCCTTGGCTGAAGCATTTCCAGCCCTCAAGCCATACGTGACG |
| | PsiM | GTCAATCCTGACAAGACGACCACTATCGATTTCGCAATACCA |
| | Nucleotide | GAAGCTCAAAGACTGTATACAGCAGCCCTCCTCTACCGCGAT |
| | Sequence | TTCGGTTTGACAATCACACTACCCCCAGATCGTTTGTGTCCA |
| | | ACGGTGCCCAATCGGCTCAACTACGTCCTCTGGATCCAGGAT |
| | | ATCCTTCAAATCACTTCTGCTGCTCTAGGTCTCCCCGAGGCA |
| | | CGTCAGGTCAAAGGGGTTGATATCGGGACCGGTGCAGCAGC |
| | | AATTTATCCCATCCTCGGTTGCTCTCTGGCCAAGAACTGGTC |
| | | CATGGTTGGAACAGAAGTAGAACAGAAGTGCATTGACATAG |
| | | CGCGCCAGAACGTCATATCTAACGGGCTCCAAGATCGTATC |
| | | ACGATAACGGCCAACACCATCGATGCGCCTATCCTTCTCCCA |
| | | CTTTTTGAGGGCGACTCTAACTTTGAGTGGGAGTTCACCATG |
| | | TGTAATCCGCCTTTTTACGACGGAGCCGCCGACATGGAGACG |
| | | TCGCAGGATGCGAAAGGCTTTGGGTTCGGAGTGAATGCTCC |
| | | ACATACAGGGACAGTTGTCGAGATGGCCACTGATGGAGGCG |
| | | AAGCCGCTTTCGTGAGCCAGATGGTTCGCGAAAGTCTCCATC |
| | | TTAAAACACGCTGCAGGTGGTTCACGAGTAACTTGGGAAAG |
| | | CTGAAGTCTCTTCATGAAATTGTGGGGCTCTTACGCGAACAT |
| | | CAGATAACCAACTACGCAATCAATGAATATGTCCAAGGAAC |
| | | TACACGCCGTTACGCAATTGCGTGGTCGTTCACCGACCTTCG |
| | | CCTTAGCGATCATTTGCCTCGCCCCCCTAATCCCGATTIGAG |
| | | TGCTTTGTTCTAG |
| 24 | Gymnopilus | MHSRNPYRSPPDFAALSAAYPPLSPYITTDLSSGRKTIDFRNEEA |
| | junonius | QRRLTEAIMLRDFGVVLNIPSNRLCPPVPNRMNYVLWIQDIVYA |
| | PsiM | HQTILGVSSRRIRGLDIGTGATAIYPILACKKEQSWEMVATELD |
| | Genbank | DYSYECACDNVSSNNMQTSIKVKKASVDGPILFPVENQNFDFS |
| | Accession No. | MCNPPFYGSKEEVAQSAESKELPPNAVCTGAEIEMIFSQGGEEG |
| | KAF8878011.1 | FVGRMVEESERLQTRCKWYTSMLGKMSSVSTIVQALRARSIMN |
| | Amino Acid | YALTEFVQGQTRRWAIAWSFSDTHLPDAVSRISS |
| | Sequence | |
| 25 | Gymnopilus | ATGCACTCTCGTAACCCTTATAGATCCCCTCCTGATTTCGCG |
| | junonius | GCATTAAGTGCGGCTTATCCTCCGCTGTCACCATACATAACT |
| | PsiM | ACCGATCTAAGCAGCGGTCGTAAAACAATTGACTTTAGAAA |
| | Nucleotide | TGAGGAAGCGCAACGTCGTCTAACTGAGGCTATCATGTTGC |
| | Sequence | GTGACTTCGGCGTTGTGTTAAACATACCATCTAACAGGCTGT |
| | (modified for | GCCCGCCTGTGCCGAATCGTATGAACTATGTACTTTGGATAC |
| | improved | AAGATATAGITTACGCGCACCAGACAATACTGGGAGTGAGT |
| | manipulation- | TCTCGTCGTATCAGAGGTCTTGATATTGGTACTGGTGCTACC |
| | silent | GCTATATATCCTATACTGGCATGCAAGAAAGAGCAGAGCTG |
| | mutations) | GGAGATGGTTGCAACTGAATTGGACGACTACTCCTATGAGT |
| | | GTGCATGTGATAACGTGTCATCCAACAATATGCAGACTTCCA |
| | | TTAAAGTAAAGAAGGCTTCGGTAGATGGGCCGATCCTGTTCC |
| | | CAGTGGAAAACCAAAATTTCGACTTTAGCATGTGCAACCCG |
| | | CCTTTCTACGGCTCTAAGGAGGAGGTGGCGCAATCCGCAGA |
| | | GTCAAAAGAACTGCCGCCCAATGCTGTTTGCACGGGTGCAG |
| | | AGATCGAGATGATATTTAGTCAAGGAGGAGAAGAGGGTTTC |
| | | GTAGGTAGAATGGTAGAGGAATCAGAGAGGTTGCAAACGAG |
| | | ATGCAAATGGTACACTTCAATGCTTGGTAAGATGTCTAGTGT |
| | | AAGCACTATAGTTCAGGCTCTGCGTGCGAGATCAATTATGAA |
| | | TTATGCTTTGACAGAATTTGTACAAGGACAAACCCGTAGGTG |
| | | GGCGATAGCTTGGTCTTTCTCCGACACTCACTTACCGGATGC |
| | | CGTCAGTAGAATCTCCAGTTAA |
| 26 | Psilocybe | MQVLPACQSSALKTLCPSPEAFRKLGWLPTSDEVYNEFIDDLTG |
| | cyanescens | RTCNEKYSSQVTLLKPIQDFKTFIENDPIVYQEFISMFEGIEQSPT |
| | PsiD | NYHELCNMFNDIFRKAPLYGDLGPPVYMIMARIMNTQAGFSAF |
| | Genbank | TKESLNFHFKKLFDTWGLFLSSKNSRNVLVADQFDDKHYGWF |
| | Accession No. | SERAKTAMMINYPGRTFEKVFICDEHVPYHGFTSYDDFFNRRFR |
| | KY984104 | DKDTDRPVVGGVTDTTLIGAACESLSYNVSHNVQSLDTLVIKG |
| | Amino Acid | EAYSLKHLLHNDPFTPQFEHGSIIQGFLNVTAYHRWHSPVNGTI |
| | Sequence | VKIVNVPGTYFAQAPYTIGSPIPDNDRDPPPYLKSLVYFSNIAAR |
| | | QIMFIEADNKDIGLIFLVFIGMTEISTCEATVCEGQHVNRGDDLG |
| | | MFHFGGSSFALGLRKDSKAKILEKFAKPGTVIRINELVASVRK |
| 27 | Psilocybe | ATGCAGGTACTGCCCGCGTGCCAATCTTCCGCGCTTAAAACA |
| | cyanescens | TTGTGCCCATCCCCCGAGGCCTTTCGAAAGCTCGGTTGGCTC |
| | PsiD | CCTACTAGCGACGAGGTTTACAACGAATTCATCGATGACTTG |
| | Nucleotide | ACCGGTCGCACGTGCAATGAAAAGTACTCCAGCCAGGTTAC |
| | Sequence | ACTTTTGAAGCCTATCCAAGATTTCAAGACATTCATCGAGAA |
| | | TGATCCCATAGTGTATCAAGAATTTATCTCTATGTTTGAAGG |
| | | AATCGAGCAGTCTCCCACCAACTACCACGAGCTATGTAACAT |
| | | GTTCAACGACATCTTTCGCAAAGCCCCACTCTACGGCGATCT |
| | | TGGTCCTCCGGTTTACATGATCATGGCCAGAATAATGAATAC |
| | | GCAGGCGGGTTTCTCTGCGTTCACAAAAGAGAGCTTGAACTT |
| | | CCATTTCAAAAAGCTCTTCGACACCTGGGGGCTATTCCTTTC |
| | | CTCGAAAAACTCTCGAAACGTGCTTGTTGCAGACCAGTTTGA |
| | | CGATAAGCATTACGGGTGGTTCAGCGAGCGAGCCAAGACTG |
| | | CCATGATGATTAATTATCCAGGGCGTACATTCGAGAAAGTCT |
| | | TCATCTGCGACGAGCACGTTCCATACCATGGCTTCACTTCCT |
| | | ATGACGATTTCTTCAATCGCAGGTTCAGGGACAAGGATACA |
| | | GATCGGCCCGTAGTCGGTGGGGTTACTGACACCACTTTAATC |
| | | GGGGCTGCCTGTGAATCGTTGTCATATAACGTCTCTCACAAC |
| | | GTCCAGTCTCTTGACACGCTAGTCATCAAGGGAGAGGCCTAT |
| | | TCACTTAAACATCTACTTCATAACGACCCCTTCACACCGCAA |
| | | TTCGAACATGGGAGCATCATTCAAGGATTCCTAAATGTCACC |
| | | GCTTACCACCGCTGGCACTCCCCCGTCAATGGCACGATTGTG |
| | | AAGATCGTCAACGTTCCAGGTACCTACTTCGCTCAAGCTCCA |
| | | TATACAATTGGATCTCCTATCCCCGATAACGACCGCGACCCG |
| | | CCTCCTTACCTCAAGTCACTCGTATACTTCTCCAACATCGCTG |
| | | CACGGCAAATTATGTTCATCGAGGCCGACAACAAAGACATC |
| | | GGCCTCATTTTCTTGGTCTTCATTGGAATGACTGAGATCTCG |
| | | ACTTGCGAGGCGACGGTGTGCGAAGGTCAGCATGTCAACCG |
| | | CGGTGACGATTIGGGCATGTTCCATTTCGGTGGTTCATCTTTT |
| | | GCCCTTGGCTTGCGGAAGGACTCGAAGGCGAAGATTTTGGA |
| | | AAAGTTCGCGAAACCGGGGACCGTTATTAGGATCAACGAGC |
| | | TAGTTGCATCTGTAAGGAAGTAG |
| 28 | Psilocybe | MTFDLKTEEGLLSYLTKHLSLDVAPNGVKRLSGGFVNVTWRV |
| | cyanescens | GLNAPYHGHTSIILKHAQPHLSSDIDFKIGVERSAYEYQALKIVS |
| | PsiK | ANSSLLGSSDIRVSVPEGLHYDVVNNALIMQDVGTMKTLLDYV |
| | Genbank | TAKPPISAEIASLVGSQIGAFIARLHNLGRENKDKDDFKFFSGNI |
| | Accession No. | VGRTTADQLYQTIIPNAAKYGIDDPILPIVVKELVEEVMNSEETL |
| | KY984102 | IMADLWSGNILLQFDENSTELTRIWLVDWELCKYGPPSLDMGY |
| | Amino Acid | FLGDCFLVARFQDQLVGTSMRQAYLKSYARNVKEPINYAKAT |
| | Sequence | AGIGAHLVMWTDFMKWGNDEEREEFVKKGVEAFHEANEDNR |
| | | NGEITSILVKEASRT |
| 29 | Psilocybe | ATGACTTTCGATCTCAAGACTGAAGAAGGCCTGCTCTCATAC |
| | cyanescens | CTCACAAAGCACCTATCGCTGGACGTTGCTCCCAACGGGGTG |
| | PsiK | AAACGTCTTAGTGGAGGCTTCGTCAACGTTACCTGGCGGGTC |
| | Nucleotide | GGGCTCAATGCCCCTTATCATGGTCACACGAGCATTATTCTG |
| | Sequence | AAGCATGCTCAACCGCACCTGTCTTCAGACATAGATTTCAAG |
| | | ATAGGTGTTGAACGATCGGCGTACGAGTATCAAGCGCTCAA |
| | | AATCGTGTCAGCCAATAGCTCCCTTCTAGGCAGCAGCGATAT |
| | | TCGGGTCTCTGTACCAGAAGGTCTTCACTACGACGTCGTTAA |
| | | TAACGCATTGATCATGCAAGATGTCGGGACAATGAAGACCC |
| | | TGTTGGACTATGTCACTGCCAAACCACCAATTTCTGCAGAGA |
| | | TCGCCAGTCTCGTAGGCAGTCAAATTGGTGCATTTATCGCTA |
| | | GGCTGCACAACCTCGGCCGCGAGAATAAAGACAAGGACGAC |
| | | TTCAAGTTCTTCTCTGGAAACATCGTCGGGAGAACAACCGCA |
| | | GACCAGTTGTATCAAACCATCATACCTAATGCCGCTAAATAC |
| | | GGTATCGACGATCCAATTCTCCCAATTGTGGTAAAGGAGTTG |
| | | GTGGAGGAGGTCATGAATAGTGAAGAAACGCTTATCATGGC |
| | | GGATTTATGGAGTGGCAATATTCTTCTCCAGTTTGATGAAAA |
| | | CTCGACGGAATTGACGAGGATATGGCTGGTAGACTGGGAGT |
| | | TGTGCAAATATGGTCCACCGTCTTTGGACATGGGGTACTTCT |
| | | TAGGCGACTGTTTCCTGGTCGCTCGATTTCAAGATCAGCTCG |
| | | TAGGGACATCAATGCGACAGGCCTACTTGAAGAGCTACGCA |
| | | AGGAATGTCAAGGAGCCAATCAATTATGCAAAAGCCACCGC |
| | | AGGCATCGGCGCGCATCTCGTCATGTGGACTGATTTCATGAA |
| | | GTGGGGGAACGATGAAGAGAGGGAAGAGTTTGTTAAGAAA |
| | | GGCGTGGAAGCCTTCCATGAAGCAAATGAGGACAATAGAAA |
| | | CGGGGAGATTACGTCTATACTTGTGAAGGAAGCATCGCGCA |
| | | CTTAG |
| 30 | Psilocybe | MHIRNPYRDGVDYQALAEAFPALKPHVTVNSDNTTSIDFAVPE |
| | cyanescens | AQRLYTAALLHRDFGLTITLPEDRLCPTVPNRLNYVLWVEDILK |
| | PsiM | VTSDALGLPDNRQVKGIDIGTGASAIYPMLACSRFKTWSMVAT |
| | Genbank | EVDQKCIDTARLNVIANNLQERLAIIATSVDGPILVPLLQANSDF |
| | Accession No. | EYDFTMCNPPFYDGASDMQTSDAAKGFGFGVNAPHTGTVLEM |
| | KY984103 | ATEGGESAFVAQMVRESLNLQTRCRWFTSNLGKLKSLYEIVGL |
| | Amino Acid | LREHQISNYAINEYVQGATRRYAIAWSFIDVRLPDHLSRPSNPD |
| | Sequence | LSSLF |
| 31 | Psilocybe | ATGCATATCAGGAACCCATACCGCGATGGTGTTGACTACCA |
| | cyanescens | AGCACTCGCTGAAGCATTTCCGGCTCTCAAACCACATGTCAC |
| | PsiM | AGTAAATTCAGACAATACGACCTCCATCGACTTTGCTGTGCC |
| | Nucleotide | AGAAGCCCAAAGACTGTATACAGCTGCCCTTCTACACCGGG |
| | Sequence | ATTTCGGTCTTACGATCACACTCCCGGAAGACCGTCTTTGTC |
| | | CGACAGTGCCTAATCGGCTCAACTATGTCCTTTGGGTTGAAG |
| | | ATATCCTTAAAGTCACTTCTGATGCTCTCGGTCTTCCGGATA |
| | | ATCGTCAAGTTAAGGGGATCGATATCGGAACTGGCGCATCA |
| | | GCGATATATCCCATGCTCGCATGCTCTCGTTTTAAGACATGG |
| | | TCCATGGTTGCAACAGAGGTAGACCAGAAGTGTATTGACAC |
| | | TGCTCGTCTCAACGTCATTGCCAACAACCTCCAAGAACGTCT |
| | | CGCAATTATAGCCACCTCCGTCGATGGTCCTATACTTGTCCC |
| | | CCTCTTGCAGGCGAATTCTGATTTTGAGTACGATTTTACGAT |
| | | GTGTAATCCGCCCTTCTACGATGGGGCATCCGACATGCAGAC |
| | | ATCGGATGCTGCGAAGGGGTTTGGATTCGGTGTGAACGCTCC |
| | | GCATACCGGCACGGTGCTIGAGATGGCCACCGAGGGAGGTG |
| | | AATCGGCCTTCGTAGCCCAAATGGTCCGCGAAAGTTTGAATC |
| | | TTCAAACACGATGCAGGTGGTTCACGAGTAATTTGGGGAAA |
| | | TTGAAGTCCTTGTACGAAATTGTGGGGCTGCTGCGAGAACAT |
| | | CAGATAAGTAACTACGCAATCAACGAATACGTCCAAGGAGC |
| | | CACTCGTCGATATGCGATTGCATGGTCGTTCATCGATGTTCG |
| | | ACTGCCTGATCATTTGTCCCGTCCATCTAACCCCGACCTAAG |
| | | CTCTCTTTTCTAG |
| 32 | Psilocybe | MQVIPACNSAAIRSLCPTPESFRNMGWLSVSDAVYSEFIGELAT |
| | cubensis | RASNRNYSNEFGLMQPIQEFKAFIESDPVVHQEFIDMFEGIQDSP |
| | PsiD | RNYQELCNMFNDIFRKAPVYGDLGPPVYMIMAKLMNTRAGFS |
| | Genbank | AFTRQRLNLHFKKLFDTWGLFLSSKDSRNVLVADQFDDRHCG |
| | Accesion No. | WLNERALSAMVKHYNGRAFDEVFLCDKNAPYYGFNSYDDFFN |
| | KY984101 | RRFRNRDIDRPVVGGVNNTTLISAACESLSYNVSYDVQSLDTLV |
| | Amino Acid | FKGETYSLKHLLNNDPFTPQFEHGSILQGFLNVTAYHRWHAPV |
| | Sequence | NGTIVKIINVPGTYFAQAPSTIGDPIPDNDYDPPPYLKSLVYFSNI |
| | | AARQIMFIEADNKEIGLIFLVFIGMTEISTCEATVSEGQHVNRGD |
| | | DLGMFHFGGSSFALGLRKDCRAEIVEKFTEPGTVIRINEVVAAL |
| | | KA |
| 33 | Psilocybe | ATGCAGGTGATACCCGCGTGCAACTCGGCAGCAATAAGATC |
| | cubensis | ACTATGTCCTACTCCCGAGTCTTTTAGAAACATGGGATGGCT |
| | PsiD | CTCTGTCAGCGATGCGGTCTACAGCGAGTTCATAGGAGAGTT |
| | Nucleotide | GGCTACCCGCGCTTCCAATCGAAATTACTCCAACGAGTTCGG |
| | Sequence | CCTCATGCAACCTATCCAGGAATTCAAGGCTTTCATTGAAAG |
| | | CGACCCGGTGGTGCACCAAGAATTTATTGACATGTTCGAGG |
| | | GCATTCAGGACTCTCCAAGGAATTATCAGGAACTATGTAATA |
| | | TGTTCAACGATATCTITCGCAAAGCTCCCGTCTACGGAGACC |
| | | TTGGCCCTCCCGTTTATATGATTATGGCCAAATTAATGAACA |
| | | CCCGAGCGGGCTTCTCTGCATTCACGAGACAAAGGTTGAAC |
| | | CTTCACTTCAAAAAACTTTTCGATACCTGGGGATTGTTCCTG |
| | | TCTTCGAAAGATTCTCGAAATGTTCTTGTGGCCGACCAGTTC |
| | | GACGACAGACATTGCGGCTGGTTGAACGAGCGGGCCTTGTC |
| | | TGCTATGGTTAAACATTACAATGGACGCGCATTTGATGAAGT |
| | | CTTCCTCTGCGATAAAAATGCCCCATACTACGGCTTCAACTC |
| | | TTACGACGACTTCTTTAATCGCAGATTTCGAAACCGAGATAT |
| | | CGACCGACCTGTAGTCGGTGGAGTTAACAACACCACCCTCAT |
| | | TTCTGCTGCTTGCGAATCACTTTCCTACAACGTCTCTTATGAC |
| | | GTCCAGTCTCTCGACACTTTAGTTTTCAAAGGAGAGACTTAT |
| | | TCGCTTAAGCATTTGCTGAATAATGACCCTTTCACCCCACAA |
| | | TTCGAGCATGGGAGTATTCTACAAGGATTCTTGAACGTCACC |
| | | GCTTACCACCGATGGCACGCACCCGTCAATGGGACAATCGT |
| | | CAAAATCATCAACGTTCCAGGTACCTACTTTGOGCAAGCCCC |
| | | GAGCACGATTGGCGACCCTATCCCGGATAACGATTACGACC |
| | | CACCTCCTTACCTTAAGTCTCTTGTCTACTTCTCTAATATTGC |
| | | CGCAAGGCAAATTATGTTTATTGAAGCCGACAACAAGGAAA |
| | | TTGGCCTCATTTTCCTTGTGTTCATCGGCATGACCGAAATCTC |
| | | GACATGTGAAGCCACGGTGTCCGAAGGTCAACACGTCAATC |
| | | GTGGCGATGACTTGGGAATGTTCCATTTCGGTGGTTCTTCGT |
| | | TCGCGCTTGGTCTGAGGAAGGATTGCAGGGCAGAGATCGTT |
| | | GAAAAGTTCACCGAACCCGGAACAGTGATCAGAATCAACGA |
| | | AGTCGTCGCTGCTCTAAAGGCTTAG |
| 34 | Psilocybe | MAFDLKTEDGLITYLTKHLSLDVDTSGVKRLSGGFVNVTWRIK |
| | cubensis | LNAPYQGHTSIILKHAQPHMSTDEDFKIGVERSVYEYQAIKLM |
| | PsiK | MANREVLGGVDGIVSVPEGLNYDLENNALIMQDVGKMKTLLD |
| | Genbank | YVTAKPPLATDIARLVGTEIGGFVARLHNIGRERRDDPEFKFFS |
| | Accesion No. | GNIVGRTTSDQLYQTIIPNAAKYGVDDPLLPTVVKDLVDDVMH |
| | KY984099 | SEETLVMADLWSGNILLQLEEGNPSKLQKIYILDWELCKYGPAS |
| | Amino Acid | LDLGYFLGDCYLISRFQDEQVGTTMRQAYLQSYARTSKHSINY |
| | Sequence | AKVTAGIAAHIVMWTDFMQWGSEEERINFVKKGVAAFHDARG |
| | | NNDNGEITSTLLKESSTA |
| 35 | Psilocybe | ATGGCGTTCGATCTCAAGACTGAAGACGGCCTCATCACATAT |
| | cubensis | CTCACTAAACATCTTTCTTTGGACGTCGACACGAGCGGAGTG |
| | PsiK | AAGCGCCTTAGCGGAGGCTTTGTCAATGTAACCTGGCGCATT |
| | Nucleotide | AAGCTCAATGCTCCTTATCAAGGTCATACGAGCATCATCCTG |
| | Sequence | AAGCATGCTCAGCCGCACATGTCTACGGATGAGGATTTTAA |
| | | GATAGGTGTAGAACGTTCGGTTTACGAATACCAGGCTATCA |
| | | AGCTCATGATGGCCAATCGGGAGGTTCTGGGAGGCGTGGAT |
| | | GGCATAGTTTCTGTGCCAGAAGGCCTGAACTACGACTTAGA |
| | | GAATAATGCATTGATCATGCAAGATGTCGGGAAGATGAAGA |
| | | CCCTTTTAGATTATGTCACCGCCAAACCGCCACTTGCGACGG |
| | | ATATAGCCCGCCTTGTTGGGACAGAAATTGGGGGGTTCGTTG |
| | | CCAGACTCCATAACATAGGCCGCGAGAGGCGAGACGATCCT |
| | | GAGTTCAAATTCTTCTCTGGAAATATTGTCGGAAGGACGACT |
| | | TCAGACCAGCTGTATCAAACCATCATACCCAACGCAGCGAA |
| | | ATATGGCGTCGATGACCCCTTGCTGCCTACTGTGGTTAAGGA |
| | | CCTTGTGGACGATGTCATGCACAGCGAAGAGACCCTTGTCAT |
| | | GGCGGACCTGTGGAGTGGAAATATTCTTCTCCAGTTGGAGG |
| | | AGGGAAACCCATCGAAGCTGCAGAAGATATATATCCTGGAT |
| | | TGGGAACTTTGCAAGTACGGCCCAGCGTCGTTGGACCTGGG |
| | | CTATTTCTTGGGTGACTGCTATTTGATATCCCGCTTTCAAGAC |
| | | GAGCAGGTCGGTACGACGATGCGGCAAGCCTACTTGCAAAG |
| | | CTATGCGCGTACGAGCAAGCATTCGATCAACTACGCCAAAG |
| | | TCACTGCAGGTATTGCTGCTCATATTGTGATGTGGACCGACT |
| | | TTATGCAGTGGGGGAGCGAGGAAGAAAGGATAAATTTTGTG |
| | | AAAAAGGGGGTAGCTGCCTTTCACGACGCCAGGGGCAACAA |
| | | CGACAATGGGGAAATTACGTCTACCTTACTGAAGGAATCAT |
| | | CCACTGCGTAA |
| 36 | Psilocybe | MHIRNPYRTPIDYQALSEAFPPLKPFVSVNADGTSSVDLTIPEAQ |
| | cubensis | RAFTAALLHRDFGLTMTIPEDRLCPTVPNRLNYVLWIEDIFNYT |
| | PsiM | NKTLGLSDDRPIKGVDIGTGASAIYPMLACARFKAWSMVGTEV |
| | Genbank | ERKCIDTARLNVVANNLQDRLSILETSIDGPILVPIFEATEEYEYE |
| | Accesion No. | FTMCNPPFYDGAADMQTSDAAKGFGFGVGAPHSGTVIEMSTE |
| | KY984100 | GGESAFVAQMVRESLKLRTRCRWYTSNLGKLKSLKEIVGLLKE |
| | Amino Acid | LEISNY AINEYVQGSTRRYAVAWSFTDIQLPEELSRPSNPELSSL |
| | Sequence | F |
| 37 | Psilocybe | ATGCATATCAGAAATCCTTACCGTACACCAATTGACTATCAA |
| | cubensis | GCACTTTCAGAGGCCTTCCCTCCCCTCAAGCCATTTGTGTCT |
| | PsiM | GTCAATGCAGATGGTACCAGTTCTGTTGACCTCACTATCCCA |
| | Nucleotide | GAAGCCCAGAGGGCGTTCACGGCCGCTCTTCTTCATCGTGAC |
| | Sequence | TTCGGGCTCACCATGACCATACCAGAAGACCGTCTGTGCCCA |
| | | ACAGTCCCCAATAGGTTGAACTACGTTCTGTGGATTGAAGAT |
| | | ATTTTCAACTACACGAACAAAACCCTOGGCCTGTCGGATGAC |
| | | CGTCCTATTAAAGGCGTTGATATTGGTACAGGAGCCTCCGCA |
| | | ATTTATCCTATGCTTGCCTGTGCTCGGTTCAAGGCATGGTCT |
| | | ATGGTTGGAACAGAGGTCGAGAGGAAGTGCATTGACACGGC |
| | | CCGCCTCAATGTCGTCGCGAACAATCTCCAAGACCGTCTCTC |
| | | GATATTAGAGACATCCATTGATGGTCCTATTCTCGTCCCCAT |
| | | TTTCGAGGCGACTGAAGAATACGAATACGAGTTTACTATGTG |
| | | TAACCCTCCATICTACGACGGTGCTGCCGATATGCAGACTTC |
| | | GGATGCTGCCAAAGGATTTGGATTTGGCGTGGGCGCTCCCCA |
| | | TTCTGGAACAGTCATCGAAATGTCGACTGAGGGAGGTGAAT |
| | | CGGCTTTCGTCGCTCAGATGGTCCGTGAGAGCTTGAAGCTTC |
| | | GAACACGATGCAGATGGTACACGAGTAACTTGGGAAAGCTG |
| | | AAATCCTTGAAAGAAATAGTGGGGCTGCTGAAAGAACTTGA |
| | | GATAAGCAACTATGCCATTAACGAATACGTTCAGGGGTCCA |
| | | CACGTCGTTATGCCGTTGCGTGGTCTTTCACTGATATTCAACT |
| | | GCCTGAGGAGCTTTCTCGTCCCTCTAACCCCGAGCTCAGCTC |
| | | TCTTTTCTAG |
| 38 | Panaeolus | MQVLTACYTSTLKSLLPSFDAFRSMGWLPVSDKTYNEWIGDLR |
| | cyanescens | SRASDKNYTSQVGLIQPIKDFKAFIESDPVVHQEFITMFEGIEESP |
| | PsiD | RNYEELCHMENDIFRKAPVYGDLGPPVYMVMARIMNTQAGFS |
| | Genbank | AFTKQSLNSHFKRLFDTWGVFLSSKESRYVLVTDQFDDNHYG |
| | Accesion No. | WLSDRAKSAMVKHYYGRTFEQVFICDEHAPYHGFQSYDDFFN |
| | PPQ80975 | RRFRDRDIDRPVVGGIENTTLISAACESLSYNVCHDLQSLDTLFV |
| | Amino Acid | KGESYSLKHLLNDDPFARQFEHGSILQGFLNVTAYHRWHAPVN |
| | Sequence | GTILKIINVPGTYFAQAPHTIGDSLDSDHPPYLKSLAYFSNIAAR |
| | | QIMFIEADNKDIGLIFLVFIGMTEISTCEATVSEGQHVNRGDDLG |
| | | MFHFGGSSFALGLRKDCKAEIFERFAEQGTVIKINEVVAAVKD |
| 39 | Panaeolus | ATGCAGGTACTGACCGCGTGCTACACTTCCACGCTTAAATCT |
| | cyanescens | TTACTCCCAAGTTTTGATGCCTTTCGAAGCATGGGATGGCTG |
| | PsiD | CCCGTCAGCGACAAGACATACAACGAATGGATAGGCGACTT |
| | Nucleotide | GAGGAGCCGCGCATCCGACAAAAACTACACCAGTCAGGTTG |
| | Sequence | GCCTCATACAGCCCATCAAGGACTTTAAAGCTTTCATCGAAA |
| | | GCGACCCCGTCGTCCATCAAGAATTTATCACGATGTTCGAGG |
| | | GCATCGAGGAGTCTCCGAGGAATTATGAGGAGCTATGTCAC |
| | | ATGTTCAACGATATCTTTCGCAAAGCTCCCGTCTACGGAGAT |
| | | CTAGGACCCCCGGTTTACATGGTCATGGCCAGAATAATGAA |
| | | CACACAGGCTGGTTTCTCTGCGTTCACAAAACAGAGTCTGAA |
| | | TTCCCACTTCAAACGGCTCTTCGACACTTGGGGTGTTTTCCTT |
| | | TCCTCGAAAGAGTCTCGCTACGTTCTCGTGACCGACCAGTTT |
| | | GACGACAATCATTACGGCTGGCTGAGCGACCGAGCCAAATC |
| | | CGCCATGGTAAAACATTACTATGGTCGCACGTTCGAACAGGT |
| | | ATTCATTTGCGACGAGCACGCGCCATACCATGGTTTCCAGTC |
| | | ATACGACGACTTTTTCAATCGCAGATTCAGGGACAGGGATAT |
| | | TGATCGGCCTGTCGTTGGCGGCATCGAAAACACCACCCTCAT |
| | | TTCTGCCGCATGCGAATCTCTTTCCTACAACGTCTGCCACGA |
| | | TTTACAATCACTCGACACACTATTCGTCAAAGGCGAATCTTA |
| | | TTCGCTCAAGCACTTGCTCAACGACGACCCATTCGCACGGCA |
| | | ATTCGAACACGGGAGCATTCTTCAGGGATTCCTAAACGTTAC |
| | | CGCCTACCATCGATGGCACGCCCCCGTCAATGGAACCATCCT |
| | | CAAAATTATCAACGTTCCCGGTACATACTTTGCGCAAGCTCC |
| | | TCACACTATCGGCGATTCGTTAGACAGCGACCACCCTCCTTA |
| | | CCTCAAGTCTCTTGCGTACTTCTCCAACATCGCCGCCAGGCA |
| | | AATCATGTTTATCGAAGCTGACAATAAGGATATCGGCCTTAT |
| | | CTTCCTTGTCTTTATCGGGATGACCGAAATCTCCACCTGCGA |
| | | GGCGACCGTATCTGAGGGCCAGCATGTCAATCGAGGTGATG |
| | | ATTTGGGCATGTTCCACTTCGGGGGTTCATCATTCGCGCTTG |
| | | GTTTACGCAAGGACTGCAAGGCGGAGATTTTTGAAAGGTTC |
| | | GCCGAACAAGGCACTGTCATCAAAATTAACGAGGTTGTTGC |
| | | GGCTGTCAAAGATTAA |
| 40 | Panaeolus | MAFDLKTVEGLIVYLTKCLSLEVDSSGVKRLSGGFVNVTWRIR |
| | cyanescens | LNAPYQGHTSIILKHAQPHMSTDKDFKIGVERSVYEYQALKVIS |
| | PsiK | ANREALGGIDSRVSAPEGLHYDVENNALIMQDVGTLKTLMDY |
| | Genbank | VIEKPAISTEMARLIGTEIGDFVARLHSIGROKRDQPDFKFFSGNI |
| | Accesion No. | VGRTTADOLYQTILPNTAKYGIDDPLLPTVVKDLVDEAMQSEE |
| | PPQ98758 | TLIMADLWTGNILVEFEEGNLSVLKKIWLVDWELCKYGPVRLD |
| | Amino Acid | MGYFLGDCFLISRFKNEQVAKAMRQAFLQRYNRVSDTPINYSV |
| | Sequence | ATTGIAAHIVMWTDFMNWGTEEERKEYVKKGVAGIHDGRNH |
| | | NVDGEITSILMQEASTA |
| 41 | Panaeolus | ATGGCTTTCGATCTCAAGACTGTAGAGGGCCTCATCGTCTAT |
| | cyanescens | CTTACTAAATGCCTGTCTTTGGAGGTCGATTCGAGTGGCGTG |
| | PsiK | AAGCGCCTCAGCGGGGGCTTCGTAAATGTAACCTGGCGCAT |
| | Nucleotide | CAGGCTCAACGCTCCTTATCAGGGTCACACGAGCATCATCTT |
| | Sequence | GAAGCATGCTCAACCACATATGTCGACCGACAAAGATTTTA |
| | | AGATCGGCGTAGAGCGCTCGGTGTACGAGTATCAGGCCCTC |
| | | AAGGTCATATCAGCCAATCGAGAGGCCCTAGGTGGTATCGA |
| | | TAGCCGAGTATCCGCACCAGAGGGCCTTCACTACGATGTGG |
| | | AGAACAATGCCCTCATCATGCAAGATGTTGGGACGTTGAAG |
| | | ACGCTCATGGATTATGTCATAGAAAAACCGGCAATTTCGAC |
| | | GGAGATGGCCCGTCTTATCGGTACTGAGATCGGGGATTTCGT |
| | | CGCCAGACTCCATAGCATAGGCCGCCAAAAGAGAGATCAAC |
| | | CTGATTTCAAGTTTTTCTCTGGAAATATTGTCGGGAGGACAA |
| | | CTGCAGATCAACTTTATCAGACTATTCTACCCAACACGGCAA |
| | | AATATGGCATTGACGACCCTCTTCTCCCCACTGTGGTGAAAG |
| | | ACCTGGTTGATGAAGCCATGCAGAGCGAAGAAACACTTATT |
| | | ATGGCAGATCTGTGGACTGGAAACATTCTCGTGGAATTOGA |
| | | GGAAGGTAATCTATCGGTATTGAAGAAGATATGGCTCGTGG |
| | | ACTGGGAGTIGTGCAAGTATGGGCCCGTGAGGTTGGATATG |
| | | GGGTATTTCTTGGGCGATTGTTTCTTGATCTCTCGATTCAAGA |
| | | ACGAGCAAGTCGCAAAGGCAATGCGACAAGCTTTCCTGCAA |
| | | CGTTATAATCGAGTTTCTGATACACCGATCAACTACTCCGTT |
| | | GCGACGACTGGCATCGCTGCCCACATCGTTATGTGGACTGAC |
| | | TTTATGAACTGGGGCACAGAAGAGGAAAGGAAAGAGTACGT |
| | | GAAGAAAGGTGTCGCAGGAATCCATGACGGGCGAAACCACA |
| | | ACGTAGATGGGGAGATTACGTCCATTCTAATGCAGGAAGCA |
| | | TCGACGGCGTAG |
| 42 | Panaeolus | MHNRNPYRDVIDYQALAEAYPPLKPHVTVNADNTASIDLTIPE |
| | cyanescens | VQRQYTAALLHRDFGLTITLPEDRLCPTVPNRLNYVLWIEDIFQ |
| | PsiM | CTNKALGLSDDRPVKGVDIGTGASAIYPMLACARFKQWSMIAT |
| | Genbank | EVERKCIDTARLNVLANNLQDRLSILEVSVDGPILVPIFDTFERA |
| | Accesion No. | TSDYEFEFTMCNPPFYDGAADMQTSDAAKGFGFGVNAPHSGT |
| | PPQ80976 | VIEMATEGGEAAFVAQMVRESMKLQTRCRWFTSNLGKLKSLH |
| | Amino Acid | EIVALLRESQITNYAINEYVQGTTRRYALAWSFTDIKLTEELYRP |
| | Sequence | SNPELGPLCSTFV |
| 43 | Panaeolus | ATGCACAACAGAAACCCATACCGCGATGTTATCGACTACCA |
| | cyanescens | AGCTCTGGCTGAGGCGTATCCGCCCCTCAAGCCACATGTGAC |
| | PsiM | TGTCAATGCTGACAATACGGCATCCATCGACCTCACCATCCC |
| | Nucleotide | AGAAGTGCAAAGGCAATATACAGCTGCACTTCTTCATCGTG |
| | Sequence | ACTTCGGTCTGACGATTACACTCCCAGAAGACCGTCTTTGCC |
| | | CAACAGTGCCAAACAGGCTGAACTATGTCCTTTGGATTGAG |
| | | GACATCTTCCAGTGCACTAATAAGGCTCTTGGTCTCTCAGAT |
| | | GACCGTCCTGTCAAAGGCGTTGACATAGGAACTGGTGCCTC |
| | | AGCAATCTATCCTATGCTGGCCTGTGCGCGTTTCAAGCAATG |
| | | GTCCATGATTGCAACAGAGGTCGAACGCAAATGTATTGACA |
| | | CGGCCCGTTTGAACGTCTTGGCCAACAATCTCCAAGACCGTC |
| | | TCTCTATCTTGGAGGTTTCCGTCGATGGTCCTATCCTTGTTCC |
| | | CATCTTCGACACTTTCGAAAGGGCAACCTCGGACTACGAGTT |
| | | CGAGTTCACGATGTGTAACCCCCCTTICTACGATGGTGCAGC |
| | | TGACATGCAAACTTCCGATGCCGCAAAAGGCTTTGGATTTGG |
| | | GGTGAATGCGCCACATTCCGGAACTGTGATCGAAATGGCCA |
| | | CTGAGGGAGGTGAAGCGGCCTTTGTCGCCCAAATGGTTCGT |
| | | GAAAGCATGAAACTTCAAACACGATGCAGATGGTTCACGAG |
| | | CAACTTGGGAAAGTTGAAGTCCTTGCATGAGATAGTGGCTCT |
| | | CCTGAGGGAATCTCAGATCACTAACTACGCAATCAATGAGT |
| | | ATGTCCAAGGGACCACTCGTCGCTACGCTCTTGCTTGGTCTT |
| | | TTACCGATATTAAATTGACTGAGGAATTGTACCGCCCATCTA |
| | | ACCCTGAATTGGGTCCTCTTTGCTCGACCTTTGTCTGA |

All publications and patents referred to herein are incorporated by reference. Various modifications and variations of the described subject matter will be apparent to those skilled in the art without departing from the scope and spirit of the invention. Although the invention has been described in connection with specific embodiments, it should be understood that the invention as claimed should not be unduly limited to these embodiments. Indeed, various modifications for carrying out the invention are obvious to those skilled in the art and are intended to be within the scope of the following claims.

1. A method for the production of psilocybin or an intermediate or a side product thereof comprising:
contacting a prokaryotic host cell with one or more expression vectors, wherein each expression vector comprises a psilocybin production gene selected from the group consisting of psiD, psiK and psiM and combinations thereof; and
culturing the host cell;
wherein at least one psilocybin production gene is from Psilocybe cyanescens, Panaeolus cyanescens, Gymnopilus dilepis, or Gymnopilus junonius.

2. The method of claim 1, wherein the psiD gene encodes a polypeptide comprising the amino acid sequence of SEQ ID NO: 18, 26, 32, or 38, or a sequence having at least 60% sequence identity thereto.

3. The method of claim 1, wherein the psiK gene encodes a polypeptide comprising the amino acid sequence of SEQ ID NO: 20, 28, 34, or 40, or a sequence having at least 60% sequence identity thereto.

4. The method of claim 1, wherein the psiM gene encodes a polypeptide comprising the amino acid sequence of SEQ ID NO: 22, 24, 30, 36, or 42, or a sequence having at least 60% sequence identity thereto.

5. The method of claim 1, wherein the host cell is selected from the group consisting of Escherichia coli, Corynebacterium glutamicum, Vibrio natriegens, Bacillus subtilis, Bacillus megaterium, Escherichia coli Nissle 1917, Clostridium acetobutlyicum, Streptomyces coelicolor, Lactococcus lactis, Pseudomonas putida, Streptomyces clavuligerus, and Streptomyces venezuelae.

6. The method of claim 1, wherein the host cell is contacted with an expression vector comprising a psiD gene, a psiK gene and a psiM gene, wherein each gene:
is under the control of a single promoter in operon configuration;
is under control of a separate promoter in pseudooperon configuration; or
is under control of a separate promoter in monocistronic configuration.

7. The method of claim 6, wherein the promoter is selected from the group consisting of G6 mutant T7, H9 mutant T7, H10 mutant T7, C4 mutant T7, consensus T7, Lac, Lac UV5, tac, trc, GAP, and xylA promoter.

8-10. (canceled)

11. The method of claim 1, wherein the intermediate or side product of psilocybin is norbaeocystin, baeocystin, 4-hydroxytryptophan, 4-hydroxytryptamine, aeruginascin, psilocin, norpsilocin, or 4-hydroxy-N,N,N-trimethyltryptamine (4-OH-TMT).

12. The method of claim 1, wherein the host cell is cultured with a supplement independently selected from the group consisting of 4-hydroxyindole, serine, methionine and combinations thereof.

13. The method of claim 12, wherein the supplement is fed continuously to the host cell.

14. The method of claim 1, wherein the host cell is grown in an actively growing culture.

15. A recombinant prokaryotic cell comprising one or more expression vectors, wherein each expression vector comprises a psilocybin production gene selected from the group consisting of psiD, psiK and psiM and combinations thereof;
wherein at least one psilocybin production gene is from Psilocybe cyanescens, Panaeolus cyanescens, Gymnopilus dilepis, or Gymnopilus junonius.

16. The recombinant prokaryotic cell of claim 15, wherein the psiD gene encodes a polypeptide comprising the amino acid sequence of SEQ ID NO: 18, 26, 32, or 38, or a sequence having at least 60%, sequence identity thereto.

17. The recombinant prokaryotic cell of claim 15, wherein the psiK gene encodes a polypeptide comprising the amino acid sequence of SEQ ID NO: 20, 28, 34, or 40, or a sequence having at least 60% sequence identity thereto.

18. The recombinant prokaryotic cell of claim 15, wherein the psiM gene encodes a polypeptide comprising the amino acid sequence of SEQ ID NO: 22, 24, 30, 36, or 42, or a sequence having at least 60% sequence identity thereto.

19. The recombinant prokaryotic cell of claim 15, wherein the prokaryotic cell is selected from the group consisting of Escherichia coli, Corynebacterium glutamicum, Vibrio natriegens, Bacillus subtilis, Bacillus megaterium, Escherichia coli Nissle 1917, Clostridium acetobutlyicum, Streptomyces coelicolor, Lactococcus lactis, Pseudomonas putida, Streptomyces clavuligerus, and Streptomyces venezuelae.

20. The recombinant prokaryotic cell of claim 15, wherein the expression vector comprises a psiD gene, a psiK gene and a psiM gene, wherein each gene:
is under the control of a single promoter in operon configuration;
is under the control of a separate promoter in pseudooperon configuration: or
is under the control of a separate promoter in monocistronic configuration.

21. The recombinant prokaryotic cell of claim 20, wherein the promoter is selected from the group consisting of G6 mutant T7, H9 mutant T7, H10 mutant T7, C4 mutant T7, consensus T7, Lac, Lac UV5, tac, trc, GAP, and xylA promoter.

22-24. (canceled)

25. An expression vector comprising a psiD gene, a psiK gene and a psiM gene;
wherein at least one of the psiD gene, the psiK gene, or the psiM gene is from Psilocybe cyanescens, Panaeolus cyanescens, Gymnopilus dilepis, or Gymnopilus junonius;
wherein each gene:
is under the control of a single promoter in operon configuration;
is under the control of a separate promoter in pseudooperon configuration: or
is under the control of a separate promoter in monocistronic configuration.

26. The expression vector of claim 25, wherein the promoter is selected from the group consisting of G6 mutant T7, H9 mutant T7, H10 mutant T7, C4 mutant T7, consensus T7, Lac, Lac UV5, tac, trc, GAP, and xylA promoter.

27-58. (canceled)
